(19) United States
(12) Patent Application Publication
(10) Pub. No.: US 2026/0263290 A1
(43) Pub. Date: Sep. 10, 2026

(54) REUSABLE OUTER COVER FOR AN ABSORBENT ARTICLE

(71) Applicant: Essity Hygiene and Health Aktiebolag, Göteborg (SE)

(72) Inventors: Lina AUGUSTSSON, Göteborg (SE); Henrik CARLÉN, Göteborg (SE); Annika HERMENIUS, Göteborg (SE)

(73) Assignee: Essity Hygiene and Health Aktiebolag, Göteborg (SE)

(21) Appl. No.: 19/166,209

(22) PCT Filed: Mar. 20, 2024
(86) PCT No.: PCT/EP2024/057410

§ 371 Date: Sep. 17, 2025

(30) Foreign Application Priority Data
Mar. 20, 2023 (WO) PCT/EP2023/057066
Mar. 20, 2023 (WO) PCT/EP2023/057067

(51) Int. Cl. A61F 13/49; A61F 13/514

(52) U.S. Cl. CPC A61F 13/49003; A61F 2013/51468

(57) ABSTRACT

A reusable outer cover for forming an absorbent article, wherein an absorbent insert may be detachably attached to the cover, is disclosed. The cover includes first and second side portions extending transversally on opposite sides of a chassis portion. The first and second side portions are permanently attached to the chassis portion along first and second side attachment segments, respectively, thus defining a side portion length. The chassis portion includes a main member and a flap member. The flap member is arranged with an outward facing side facing an inward facing side of the main member to form a pocket. The flap member is permanently attached to the main member along first and second flap attachment segments of the second chassis side edge which define a waist region length. The side portion length extends by a longitudinal distance beyond the waist region length in the direction towards the transversal axis.

### FIELD OF THE INVENTION

The present invention relates to a reusable outer cover for an absorbent article, wherein, in the absorbent article, an absorbent insert is detachably attached to the outer cover. Also, the present invention relates to an absorbent article comprising a reusable outer cover and an absorbent insert detachably attached to the outer cover.

### BACKGROUND

Absorbent articles, in the forms of diapers and incontinence pads, are generally used for babies and incontinent adults for the absorption of bodily exudates, such as blood, urine, and faeces. In particular when looking at products that are classified as heavy incontinence articles for adults, which articles have a relatively high absorption capacity, these products are often used by physically and/or mentally impaired persons. Generally, these heavy incontinence products will be handled by a caregiver to put on and remove from the wearer's body and bought by care homes, hospitals and/or care giving relatives.

Requirements put on these products may include: being leakproof, being easy to use and change, being comfortable to wear, do not negatively affect the wearer's skin health, and maintain the wearer's dignity during changing and wearing.

To meet these requirements, currently used incontinence products are still mostly disposable products, with single-use products being the standard for heavy incontinence. Disposable products are easy to use, since they require very limited handling after use. Both the production and disposal of these products, however, result in a lot of material waste. After use, the products either end up as land fill, or, in particular for adults who are on one or more heavy medications, the absorbent articles may be incinerated. There is now an increasing need for more sustainable alternatives. To this end, products have been proposed which comprises an outer cover and an absorbent insert, wherein one or both of the outer cover and the absorbent insert is/are reusable.

For this type of products, it is of importance to ensure that the outer cover is capable of ensuring that the absorbent insert is correctly positioned in relation to the wearer during use of the absorbent article, such that the security against leakage and the desired comfort is maintained. Moreover, it is of importance to ensure that the absorbent article comprising the outer cover and the absorbent insert fulfil the wearer's needs in terms of comfort, fit, and dignity.

Thus, it is an aim to provide an outer cover for an absorbent article, and/or an absorbent article that may be a sustainable alternative to current disposable products.

Further, it is an aim to provide an outer cover for an absorbent article, and/or an absorbent article which may satisfactory fulfil one or more of the above-mentioned requirements, and/or to provide an improvement or an alternative in relation to current products.

### SUMMARY

### Definitions

With “wearer” is meant a person which may be wearing an absorbent article as described herein.

With “user” is meant a person handling the absorbent article as described herein, i.e. preparing an absorbent article for use and/or applying the absorbent article to the wearer. The user may be a wearer or a caregiver to the wearer.

With “liquid permeable” is meant herein a material being liquid permeable as understood in the sense of the art of absorbent articles. As such, the material is not intended to hinder fluids from leaking through the material to any significant degree. Known liquid permeable materials are for example such as being used as topsheet materials in conventional disposable absorbent products. In another example, conventional textile materials such as woven or knitted materials are generally liquid permeable. A liquid permeable material may for example be a textile material being free from any liquid resistant coatings or treatments. A liquid permeable material may for example be a textile material which do not absorb fluid to any significant degree.

With “breathable” is meant herein a material allowing water vapor to escape through the material.

For example, materials which are breathable while still being liquid-impermeable are commonly used in absorbent articles as backsheet materials. Such backsheet materials may be breathable while still preventing liquids from passing through the backsheet material. Such breathable, liquid impermeable materials may for example be porous polymeric films, and laminates from porous polymeric films and nonwovens.

Textile materials which form apertures, such as knitted or woven textile materials, are generally breathable.

The breathability of a material is normally measured by the water vapor transmission rate. For example, a breathable material suitable for absorbent articles may have a water vapor transmission rate of at least 1000 g/m²/24 h, when measured according to the Inda and EDANA standard test method NWSP 070.5.R1 (22) at 37.7° C.

With “total absorption capacity” is meant herein the total absorption capacity, as measured according to the Rothwell method, ISO 11948.

With “use cycle” of an absorbent article is meant herein a series of subsequent steps comprising the application of the absorbent article to a wearer, the wearer wearing the article for a limited period of time (for example during one or a few urinary discharges), and the removal of the absorbent article from the wearer.

With “reusable” is meant herein an item which intended to be used for more than one use cycle. Thus, a reusable item is an item which may be used throughout a repeated number of use cycles. If required, the reusable item may be cleaned, for example washed, or otherwise restored between use cycles.

For example, a reusable item may be designed to be used for at least 50 use cycles, such as at least 100 use cycles, or at least 500 use cycles.

With “single-use” is meant herein an item which is intended to be used for only one use cycle, and then discarded. Thus, an item being configured for single-use is typically disposed after being used by a wearer for a limited period of time (for example during one or a few urinary discharges).

By “washable” it is meant that a washable item may be cleaned by laundering. The item may thus be subjected to an aqueous solution containing detergent without losing its structural features. This aqueous solution may be heated as part of the laundering process, e.g. to 40° C. or 60° C.

A washable item, such as an outer cover, should be able to be washed at least a predetermined number of times, without significantly deteriorated features. In particular, a washable outer cover should be able to be washed a predetermined number of times without significant impairment of the fastening members, such as any insert fastener members or of fastening members for fastening the outer cover around the waist of the wearer. Also, the washable outer cover should preferably be able to be washed a predetermined number of times without significant impairment of the elasticity of the leg elastics and/or other materials, such as fabrics, of the outer cover. Finally, the washable outer cover should preferably be able to be washed a predetermined number of times without significant impairment of the colour of the outer cover.

For example, it is preferred herein that a washable item, such as the washable outer cover is able to sustain at least 20 washes in 60° C., and yet more preferred that the washable outer cover is able to sustain at least 50 washes in 60° C.

Also, it is preferred that the washable item, such as the washable outer cover is “machine washable”, i.e. that the washes mentioned in the above may take place in a washing machine.

After wash, the washable item, such as the washable outer cover may be dried e.g. by line drying. Optionally, the washable item, such as the washable outer cover may be adapted for tumble drying. In this case, the washable item should be adapted to sustain tumble drying without significant impairments, similar to the requirements in the above for an item to sustain washing.

With “stretchable” is meant herein a material which may be stretched from an original measure and is able to recover to essentially the same measure, when subject to forces during normal use of the article.

With “surface fastening material” is meant herein a material which may be substantially uniformly distributed over the surface of an area, so as provide for substantially uniform fastening by the surface fastening material over the area. Thus, any portion of the area being provided with the surface fastening material will be ready for attachment via the surface fastening material.

In some examples, a surface fastening material may provide for continuous fastening over the surface of the area comprising the surface fastening material. Such a surface fastening material may for example be an adhesive, which may be applied over the area.

In some examples, a surface fastening material may provide for fastening in a plurality of discrete fastening elements, the density of the fastening elements being such that substantially uniform fastening is provided over the area comprising the surface fastening material. For example, a density of such discrete fastening elements may be at least 10 discrete fastening elements per cm2, or more preferred at least 50 discrete fastening elements per cm2.

For example, the plurality of discrete fastening elements may provide for mechanical and/or frictional fastening over the area comprising the surface fastening material. Examples of surface fastening materials comprising discrete fastening elements may be hook materials and/or loop materials for forming hook/hook type or hook/loop type connections.

With a “fastener area comprising a surface fastening material” is meant a surface area over which a surface fastening material is distributed, so as to provide for substantially uniform fastening over the fastener area.

With “detachable” fastening is meant herein that the fastener areas may be used to attach and detach to a surface of another area, such as to attach and detach to a corresponding fastener area, multiple times without substantial impairment of their function. For a fastener area on a disposable product, it may be sufficient that the fastener area provides for a few cycles of attachment/detachment, such as at least 5 cycles or at least 2 cycles. For a fastener area on a reusable product, more cycles of attachment/detachment may be required. For example, for any reusable fastener area, the fastener area may provide for at least 50 cycles of attachment/detachment, such as at least 150 times or at least 300 times.

With “complementary” surface fastening materials is meant herein that two respective surface fastening materials are matching, allowing a detachable fastening, i.e. a detachable bond to be formed therebetween. For example, mechanical and/or frictional detachable attachment may be provided between complementary surface fastening materials. An example of complementary surface fastening materials is a first surface fastening material comprising hooks, e.g. being a hook material and a second surface fastening material comprising loops, e.g. being a loop material.

“Knitting” is a method of constructing a fabric by interlocking a series of loops of one or more yarns. There are two major classes of knitting namely warp and weft knitting.

“Weaving” is a method or process of interlacing two yarns of similar materials so that they cross each other at right angles to produce woven fabric. The warp yarns run lengthwise in the fabric and the filling threads or picks, run from side to side.

Jacquard is a system of weaving or knitting that utilizes a versatile pattern mechanism to produce intricate designs by using punch cards controlling the patterns produced. Jacquard knit may be valid for circular, flatbed, warp or weft knit.

In the context of the present disclosure, a “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers, continuous single fibers, untwisted bundles of continuous filaments, and twisted bundles of continuous filaments. Nonwoven fabrics can be formed by many processes such as meltblowing, spunbonding, solvent spinning, electrospinning, and carding.

The term “nonwoven”, mentioned in relation to the different parts of the insert, which in term of their properties are located between the groups of paper and cardboard on the one hand and textiles on the other hand. As regards nonwovens, a large number of extremely varied production processes are used, such as airlaid, wetlaid, spunlaced, spunbond, meltblown techniques etc. The fibres may be in the form of endless fibres or fibres prefabricated with an endless length, as synthetic fibres produced in situ or in the form of staple fibres. Alternatively, they may be made from natural fibres or from blends of synthetic fibres and natural fibres.

In the context of the present disclosure, a longitudinal length is, unless otherwise defined, to be understood as the longest length of a specified part or sub part along a longitudinal axis. Analogously, a transversal width is, unless otherwise defined, to be understood as the widest width of a specified part or sub part along a transversal axis.

When referring to the length of a set comprising one or more items (i.e. a length spanned by a set comprising one or more items), it is referred to the maximum longitudinal length including all of the items in the set, i.e. regardless of whether the items are transversally aligned or not.

Similarly, when referring to the width of a set comprising one or more items (i.e. a width spanned by a set comprising one or more items), it is referred to the maximum transversal width including all of the items in the set, i.e. regardless of whether the items are longitudinally aligned or not.

By the term “entirely contained within” is meant herein that an item being “entirely contained within” e.g. a part or portion does not extend beyond, e.g. does not protrude from, the part or portion.

For the purposes of the present disclosure, and unless otherwise explicitly mentioned, lengths, widths and other dimensions of an absorbent hygiene product, an outer cover, or an insert are measured with the item in a flattened-out state on a flat surface with any contracting elastics being deactivated. With “deactivated” is meant that the elastics are not active to contract any parts to which it is attached. To deactivate the elastics, the elastics may for example be cut at one or more locations, or the elastics may be removed.

When the length of an active part of an elastic element along another portion is to be determined, the start and end point of the active part of the elastic element may initially be marked on the relevant portion. Then, the elastic elements may be deactivated, and the distance between the marked start and end point may be measured on the relevant portion when in a flattened-out state.

The active part of the elastic element is the part which, in use, may be active to contract any part to which it is attached. As such, using the procedure in the above, parts of an elastic element which cannot be activated, such as glued down end sections of elastic threads, are disregarded.

As used herein, the term “area” of a portion of a product corresponds to the surface area of a flat surface covered by the portion when the product is placed on the flat surface in a flattened-out state with any elastics removed or deactivated.

To determine the flat area of an item such as the outer cover, which may comprise several joined panels or sections, it may be necessary to cut the item into several flat pieces to determine the area of each piece.

### Outer Cover

In a first aspect, the present disclosure relates to an outer cover in accordance with appended claim 1.

Thus, there is provided a reusable outer cover for forming an absorbent article wherein an absorbent insert is adapted to be detachably attached to the outer cover. The outer cover has a front portion to be directed towards a front of a wearer, a back portion to be directed towards a back of a wearer, and a crotch portion extending between the front portion and the back portion. The outer cover extends along a longitudinal central axis, and along a transversal central axis, which is perpendicular to the longitudinal axis, the transversal central axis dividing the outer cover into a front half and a back half, and a thickness axis is defined perpendicular to both the longitudinal and transversal axes, wherein an inward direction is defined along the thickness axis being the direction towards a wearer when the outer cover is worn, and an outward direction is defined along the thickness axis being the direction away from the wearer when the outer cover is worn.

The outer cover comprises a chassis portion extending along the longitudinal axis from a front chassis edge forming part of the front edge of the outer cover to a back chassis edge forming part of the back edge of the outer cover. The chassis portion comprises a first chassis side edge and a second chassis side edge respectively extending between the chassis front and back edges of the chassis portion, on opposite sides with respect to the longitudinal axis. In at least one out of the back half and the front half of the outer cover, preferably at least in the back half of the outer cover, the outer cover comprises a first and a second side portion extending transversally on opposite sides of the chassis portion with respect to the longitudinal axis. Each of the side portions of the outer cover comprises one or more waist attachment elements to attach the outer cover around the waist of a wearer during use. The first side portion is permanently attached to the chassis portion along a first side attachment segment of the first chassis side edge, and the second side portion is permanently attached to the chassis portion along a second side attachment segment of the second chassis side edge. The first and second side attachment segments define a side portion length from the longitudinal ends of each segment closest to the central transversal axis and to the adjacent back edge or front edge of the outer cover.

The chassis portion comprises a main member and a flap member. The main member forms the front or back chassis edge, the first chassis side edge, and the second chassis side edge in the front or back half. The flap member being arranged with an outward facing side of the flap member facing an inward facing side of the main member so as to form a pocket between the outward facing side of the flap member and the inward facing side of the main member. The flap member is permanently attached to the main member along a first flap attachment segment of the first chassis side edge and along a second flap attachment segment of the second chassis side edge. The first and second flap attachment segments defines a waist region length from the longitudinal ends of each segment closest to the central transversal axis and to the adjacent back edge or front edge of the outer cover.

Moreover, the side portion length extends by a longitudinal distance beyond the waist region length in the direction towards the transversal axis (X).

Optionally, the first and second side attachment segments, and/or the first and second flap attachment segments extend essentially parallel to the longitudinal axis.

Optionally, the outer cover comprises a first joint assembly comprising one or more joints connecting the first side portion to the chassis portion along the first side attachment segment, and connecting the flap member to the main member along the first flap attachment segment and
a second joint assembly comprising one or more joints connecting the second side portion to the chassis portion along the second side attachment segment, and connecting the flap member to the main member along the second flap attachment segment.

Optionally, the outer cover comprises a first joint connecting the first side portion to the chassis portion along the first side attachment segment, and connecting the flap member to the main member along the first flap attachment segment and
a second joint connecting the second side portion to the chassis portion along the second side attachment segment, and connecting the flap member to the main member along the second flap attachment segment.

Optionally, each of the joint or joints, such as each of the first and second joints, is a stitched seam, a welded seam, an adhesive seam, an adhesive tape or combinations thereof.

Optionally, wherein the joint or joints extend essentially parallel to the longitudinal axis.

Optionally, the longitudinal distance is no less than 0.50× the waist region length, such as no less than 0.75× the waist region length or no less than 0.90× the waist region length.

Optionally, the longitudinal distance is at least 5 cm, such as at least 10 cm, or from 5 to 20 cm, or from 10 to 20 cm.

Optionally, the side portion length is from 20% to 30% of a central outer cover length of the outer cover, preferably from 22% to 27%, most preferred about 25% of the central outer cover length of the outer cover.

Optionally, the waist region length is less than 15% of a central outer cover length of the outer cover, preferably less than 10% of a central outer cover length of the outer cover.

Optionally, a waist region of the chassis portion is defined transversally between the first and second flap attachment segments, the waist region having the waist region length, and the waist region of the chassis portion comprises one or more insert fastener areas for detachably fastening an insert to the outer cover, preferably the insert fastener areas comprises at least two insert fastener areas being transversally spaced and located on each transversal side of the central longitudinal axis (Y).

Optionally, at least one out of the outward facing side of the flap member and the inward facing side of the main member forming the pocket comprises one or more insert fastener areas for detachably fastening an insert to the outer cover. Advantageously, at least one of the outward facing side of the flap member and the inward facing side of the main member forming the pocket comprises at least two insert fastener areas being transversally spaced and located on each transversal side of the central longitudinal axis. For example, the outward facing side of the flap member may comprise at least two insert fastener areas being transversally spaced and located on each transversal side of the central longitudinal axis.

Optionally, the chassis portion of the outer cover further comprises a first elastic edge element and a second elastic edge element, wherein
the first elastic edge element is attached along at least a part of the first chassis side edge and the first elastic edge element is adapted to
   gather a first gathered edge segment of the first chassis side edge when the first elastic edge element is in a relaxed condition and the first gathered edge segment is gathered with a gathered extension and;
   stretch out the first gathered edge segment of the first side edge when the first elastic element is in an outstretched condition and the first gathered edge segment is flat with an ungathered extension; and
the second elastic edge element is attached along at least a part of the second chassis side edge and the second elastic edge element is adapted to
   gather a second gathered edge segment of the second chassis side edge when the second elastic edge element is in a relaxed condition and the second gathered edge segment is gathered with a gathered extension and;
stretch out the second gathered edge segment of the second chassis side edge when the second elastic element is in an outstretched condition and the second gathered edge segment is flat with an ungathered extension.

Optionally, a ratio of the ungathered extension of the first and second gathered edge segments to the gathered extension of the respective first and second gathered edge segments is in the range from 2:1 to 3.5:1. For example, the ratio may be in the range 2.5:1 to 3:1.

Optionally, the first gathered edge segment and the first side attachment segment are spaced by a second longitudinal distance, and the second gathered edge segment and the second side attachment segment are spaced by a second longitudinal distance, wherein the second longitudinal distance is no more than 3 cm, preferably no more than 2 cm.

Optionally, the first gathered edge segment follows directly from the first side attachment segment, and the second gathered edge segment follows directly from the second side attachment segment.

Optionally, when the outer cover comprises a first joint assembly or first joint, and a second joint assembly or a second joint, the first joint assembly or first joint overlaps with the first elastic edge element, and the second joint assembly or second joint overlaps with the second elastic edge element, as seen in the thickness direction.

Optionally, the main member has a first transversal Cloth Percentual Stretch in a region extending transversally between the first side attachment segment and the second side attachment segment, and longitudinally along the side portion length, and the flap member and the main member together have a second transversal Cloth Percentual Stretch in a waist region extending transversally between the first flap attachment segment and the second flap attachment segment and longitudinally along the waist region length, wherein the second transversal Cloth Percentual Stretch is less than the first transversal Cloth Percentual Stretch.

In a second variant of the first aspect, the present disclosure relates to an outer cover according to claim 18.

Thus, there is provided a reusable outer cover for forming an absorbent article wherein an absorbent insert is adapted to be detachably attached to the outer cover. The outer cover has a front portion to be directed towards a front of a wearer, a back portion to be directed towards a back of a wearer, and a crotch portion extending between the front portion and the back portion. The outer cover extends along a longitudinal central axis, and along a transversal central axis, which is perpendicular to the longitudinal axis, the transversal central axis dividing the outer cover into a front half and a back half, and a thickness axis is defined perpendicular to both the longitudinal and transversal axes. The outer cover comprises a chassis portion extending along the longitudinal axis from a front chassis edge at the front edge of the outer cover to a back chassis edge at the back edge of the outer cover. The chassis portion comprises a first chassis side edge and a second chassis side edge respectively extending between the chassis front and back edges of the chassis portion, on opposite sides with respect to the longitudinal axis. In at least one out of the back half and the front half of the outer cover, preferably at least in the back half the outer cover comprises
a first and a second side portion extending transversally on opposite sides of the chassis portion with respect to the longitudinal axis to fasten the outer cover around the waist of a wearer. Each of the side portions of the outer cover comprises one or more waist attachment elements to attach the outer cover around the waist of a wearer.

The first side portion is permanently attached to the chassis portion along a first side attachment segment of the first chassis side edge, and the second side portion is permanently attached to the chassis portion along a second side attachment segment of the second chassis side edge. The first and second side attachment segments defines a side portion length from the longitudinal ends of each segment closest to the central transversal axis and to the adjacent back edge or front edge of the outer cover.

The chassis portion comprises a waist region extending transversally between the first and second side attachment segments, and longitudinally from the front or back edge of the outer cover and towards the central longitudinal axis along a waist region length. The chassis portion further comprises an intermediate region extending transversally between the first and second side attachment segments, and longitudinally from the waist region towards the central longitudinal axis along a first distance. The intermediate region has a first transversal Cloth Percentual Stretch, and the waist region has a second transversal Cloth Percentual Stretch, and the second transversal Cloth Percentual Stretch is less than the first transversal Cloth Percentual Stretch.

Optionally, in the second variant, the waist region of the chassis portion comprises one or more insert fastener areas for detachably fastening an insert to the outer cover, preferably the insert fastener areas comprises at least two insert fastener areas being transversally spaced and located on each transversal side of the central longitudinal axis.

Optionally, in the second variant, the longitudinal distance is no less than 0.50× the waist region length, such as no less than 0.75× the waist region length or no less than 0.90× the waist region length.

Optionally, in the second variant, the longitudinal distance is at least 5 cm, such as at least 10 cm, or from 5 to 20 cm, or from 10 to 20 cm.

Optionally, in the second variant, the side portion length from 20% to 30% of a central outer cover length of the outer cover, preferably from 22% to 27%, most preferred about 25%.

Optionally, in the second variant, the waist region length is less than 15% of a central outer cover length of the outer cover, preferably less than 10% of a central outer cover length of the outer cover.

Optionally, also in the second variant, the chassis portion of the outer cover further comprising a first elastic edge element and a second elastic edge element, wherein
the first elastic edge element is attached along at least a part of the first chassis side edge and the first elastic edge element is adapted to
   gather a first gathered edge segment of the first chassis side edge when the first elastic edge element is in a relaxed condition and the first gathered edge segment is gathered with a gathered extension and;
   stretch out the first gathered edge segment of the first chassis side edge when the first elastic element is in an outstretched condition and the first gathered edge segment is flat with an ungathered extension; and
the second elastic edge element is attached along at least a part of the second chassis side edge and the second elastic edge element is adapted to
   gather a second gathered edge segment of the second chassis side edge when the second elastic edge element is in a relaxed condition and the second gathered edge segment is gathered with a gathered extension and;
stretch out the second gathered edge segment of the second chassis side edge when the second elastic element is in an outstretched condition and the second gathered edge segment is flat with an ungathered extension.

Optionally, in the second variant, the first gathered edge segment and the first side attachment segment, and the second gathered edge segment and the second side attachment segment are each spaced by a second longitudinal distance, wherein the second longitudinal distance is no more than 3 cm, preferably no more than 2 cm.

Optionally, in the second variant, the first gathered edge segment follows directly from the first side attachment segment, and the second gathered edge segment follows directly from the second side attachment segment.

Optionally, also in the second variant, similar to the first variant, the chassis portion comprises a main member and a flap member,
the main member forming the front or back chassis edge, the first chassis side edge, and the second chassis side edge in the front or back half, and
the flap member being arranged with an outward facing side of the flap member facing an inward facing side of the main member so as to form a pocket between the outward facing side of the flap member and the inward facing side of the main member,
the flap member being permanently attached to the main member along a first flap attachment segment of the first chassis side edge and along a second flap attachment segment of the second chassis side edge, the first and second flap attachment segments defining the waist region length from the longitudinal ends of each segment closest to the central transversal axis and to the back edge of the back half, or the front edge of the front half, of the outer cover,
wherein the side portion length extends by the longitudinal distance beyond the waist region length in the direction towards the transversal axis (X).

Thus, in accordance with the second variant in the immediate above, this second variant may be combined with any one of the features as mentioned for the first variant, and to similar advantages.

As mentioned in the above, for the first variant, a region with a first transversal Cloth Percentual Stretch may be defined, and a region with a second transversal Cloth Percentual Stretch may be defined.

Optionally, in the first variant or in the second variant, the second transversal Cloth Percentual Stretch is from 30 to 70% of the first transversal Cloth Percentual Stretch, such as from 40 to 60% of the first transversal Cloth Percentual Stretch, such that from 45 to 55% of the first transversal Cloth Percentual Stretch.

Optionally, in the first variant or in the second variant, each of the first and the second side portions forms a side region, wherein each side region has a side portion transversal Cloth Percentual Stretch, the side portion transversal Cloth Percentual Stretch being less than the first transversal Cloth Percentual Stretch.

Optionally, in the first variant or in the second variant, the side portion transversal Cloth Percentual Stretch is from 30 to 70% of the first transversal Cloth Percentual Stretch, such as from 40 to 60% of the first transversal Cloth Percentual Stretch, such that from 45 to 55% of the first transversal Cloth Percentual Stretch.

### Absorbent Article

In a second aspect, the present disclosure relates to an absorbent article according to claim 32. Accordingly, there is provided an absorbent article comprising an outer cover according to the first aspect (the first or the second variant), and an absorbent insert detachably attached to the outer cover.

The absorbent insert may be reusable or disposable.

The absorbent insert may be liquid-impermeable, for example, the absorbent insert may comprise a liquid-impermeable backsheet.

In the following, variants of the different aspects (outer cover and absorbent article) of the disclosure will be described with reference to exemplary embodiments and to the drawings. It is to be understood that the drawings are schematical and that the items depicted are not necessarily drawn to scale.

### BRIEF DESCRIPTION OF THE DRAWINGS

Variants of an outer cover, an insert, and an absorbent article will be described by way of example, with reference to the attached drawings, in which:

FIG. 1a shows an embodiment of an outer cover according to the first aspect of the disclosure, when in a flat laid-out state and seen from an inside direction of the outer cover,

FIG. 1b is the same Figure as FIG. 1a, but with some additional reference numerals indicated.

FIG. 2 shows the outer cover of FIGS. 1a-1b, when in a flat laid-out state and seen from an outside direction of the outer cover,

FIGS. 3a-3e shows an embodiment of parts together making up the outer cover of FIGS. 1a-1b,

FIG. 4 shows an embodiment of an absorbent insert being detachably attachable to the outer cover of FIGS. 1a-1b, the insert being in a flat laid-out state and as seen from an inside direction of the insert,

FIG. 5 shows an absorbent article comprising the outer cover of FIGS. 1a-1b and the absorbent insert of FIG. 4 being detachably attached to the outer cover, and

FIGS. 6a-6d shows a use cycle of an absorbent article comprising an outer cover and an absorbent insert.

In the drawings, the same reference numbers generally denote the same features.

### DESCRIPTION OF VARIANTS

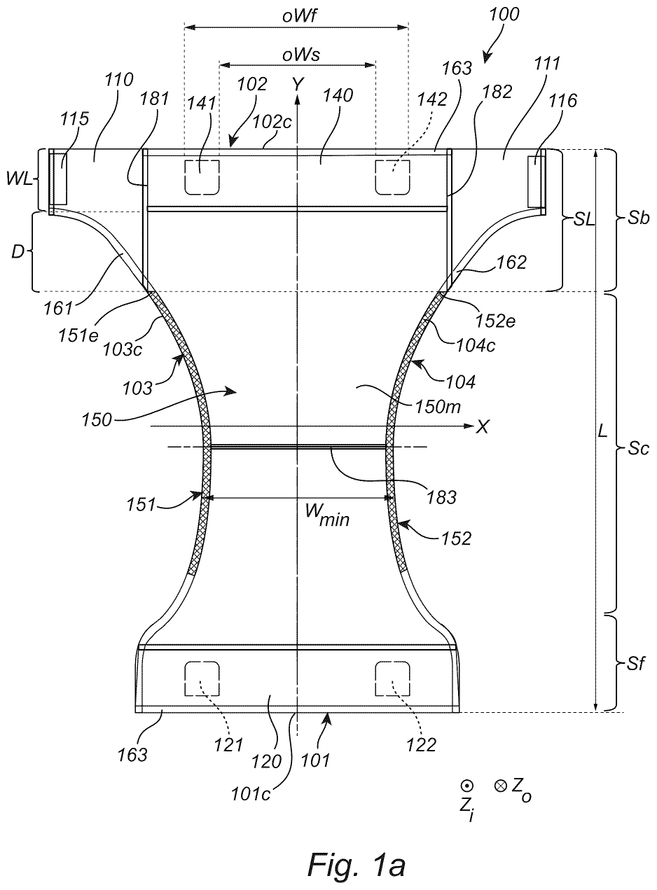

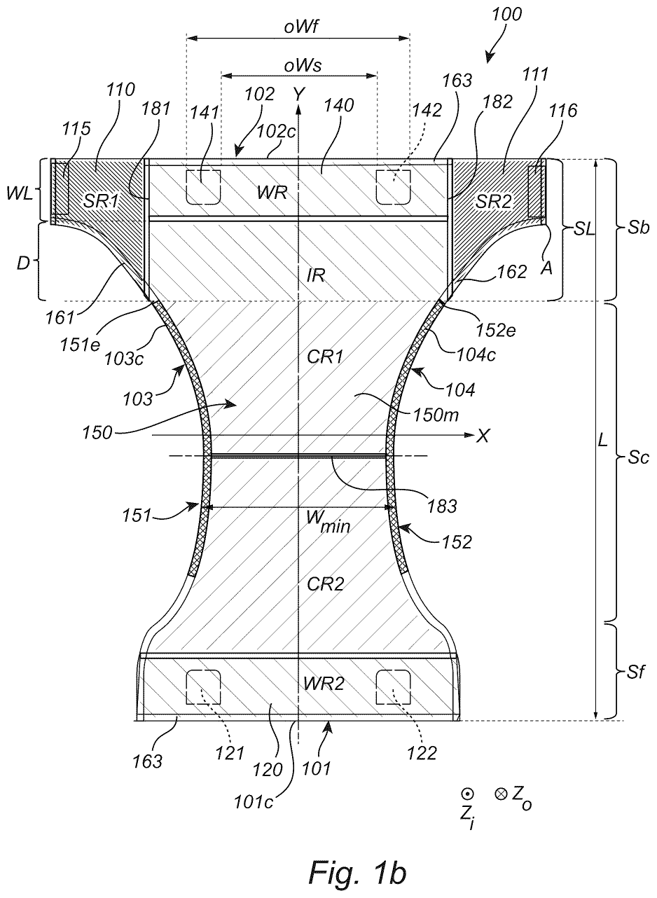

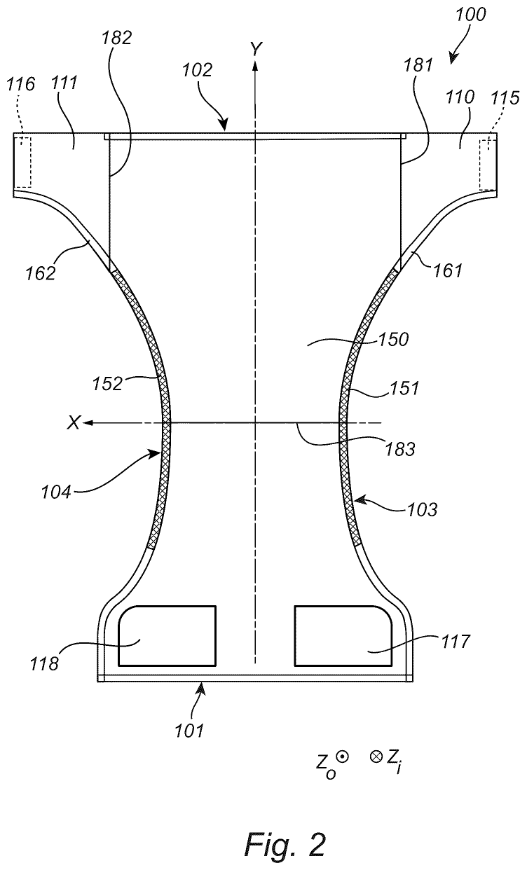

FIG. 1a shows a variant of an outer cover 100 in accordance with the present disclosure, and when in a flat laid-out state and seen from an inside direction of the outer cover. FIG. 1b is the same figure as FIG. 1a, only with some additional reference numbers. Thus, it is intended for FIG. 1a and FIG. 1b to be read together, showing the outer cover in a flat laid-out state and as seen from an inside direction of the outer cover. FIG. 2 show the outer cover 100 of FIGS. 1a-1b in the same flat laid-out state but seen from an outside direction of the outer cover.

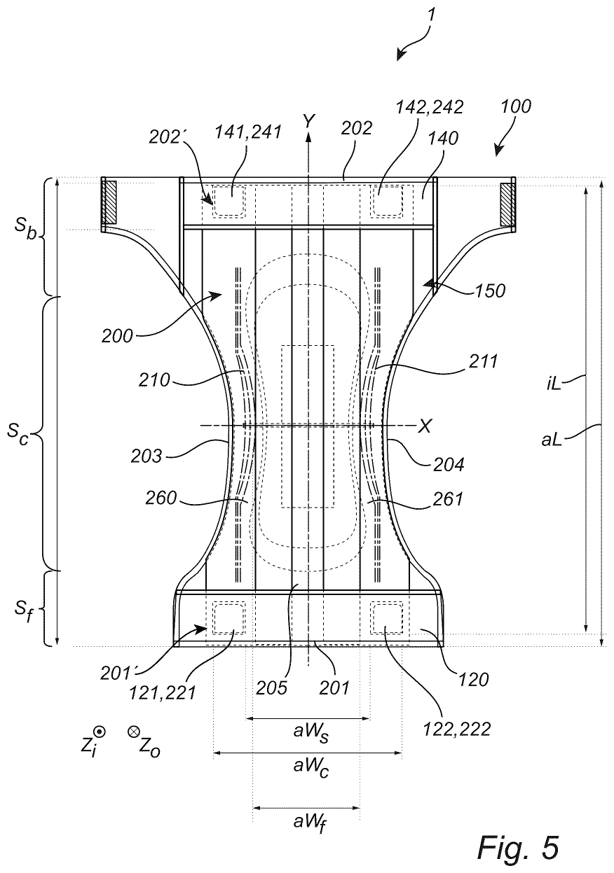

The outer cover is for forming an absorbent article 1 (See FIG. 5) wherein an absorbent insert 200 is adapted to be detachably attached to the outer cover 100.

### Properties of the Outer Cover

The outer cover is reusable, i.e. intended to be used for more than one use cycle of an absorbent article. As such, at the start of each use cycle, a new absorbent insert may be attached to the outer cover to form the absorbent article, and after each use cycle, the absorbent insert may be removed from the outer cover and disposed or cleaned. Also, the outer cover may be configured to be cleanable between use cycles. As such, the outer cover may if necessary be cleaned between each use cycle. However, it may be preferred that the outer cover is only cleaned when deemed necessary, i.e. when, after one or more use cycles, the outer cover is deemed to be soiled so as to require cleaning.

To provide for efficient cleaning, the outer cover may be configured to be washable.

For example, the outer cover may be configured to be machine-washable.

For example, the outer cover may be configured for at least 20 wash cycles, such as for at least 20 machine-wash cycles as defined herein.

Optionally, the outer cover may be configured for at least 50 wash cycles, such as for at least 50 machine-wash cycles as defined herein.

Optionally, the outer cover may be configured for at least 20 machine-wash cycles at 60 degrees Celsius, such as at least 50 machine-wash cycles at 60 degrees Celsius.

Washing allows for hygienic cleaning of the outer cover, further increasing the reusability of the cover. Being machine washable is preferred, as this makes the product easier for caregivers to clean and reuse.

As mentioned in the above, the outer cover is not primarily intended to collect bodily discharges such as urine or faeces from the wearer. Instead, the absorbent insert, which is to be attached to the outer cover when the absorbent article is in use, is to perform the function of absorbing and collecting bodily discharges from the wearer.

As such, the outer cover may be mainly liquid permeable.

The outer cover being mainly liquid permeable implies that the outer cover is substantially permeable to liquids, such as urine.

As such, at least 50% of a total area of the outer cover may be liquid permeable. For example, at least 70%, or at least 90%, of a total area of the outer cover may be liquid permeable.

That the outer cover may be mainly liquid permeable implies that suitable materials for the outer cover may be selected among liquid permeable materials.

Thus, the outer cover may be made by liquid permeable materials, such as textile fabrics. This provides for the absorbent article resulting by the combination of the outer cover and an absorbent insert having a more underwear-like look and feeling.

The outer cover may be mainly breathable. This is beneficial for the skin health of the wearer.

As such, at least 50% of a total area of the outer cover may be breathable. For example, at least 70% or at least 90% of a total area of the outer cover may be breathable.

Preferably, the outer cover may be mainly liquid permeable and mainly breathable. As such, the outer cover may be made by liquid permeable, breathable materials such as for example textile materials.

Suitably, the outer cover may be mainly liquid permeable, mainly breathable, and washable as set out in the above. To that end, suitable materials being liquid permeable, breathable, and washable may be selected to form the outer cover. Again, suitable materials may be found among textile materials being liquid permeable, breathable, and washable.

For example, the garment portion of the outer cover may comprise one or more fabric panels forming the front portion, the back portion, and the crotch portion of the garment portion.

As such, the one or more fabric panels may be selected from materials being liquid permeable, breathable, and/or washable.

For example, the one or more fabric panels may comprise a textile fabric. Suitable textile fabrics may be liquid permeable, breathable and/or washable.

For example, the one or more fabric panels may comprise a knitted textile fabric.

A knitted textile fabric may be suitable to provide elasticity to the outer cover, as discussed in the below.

For example, the one or more fabric panels may comprise a weft knit textile fabric material, for example as a weft knit single jersey.

For example, the outer cover may comprise a textile fabric, having a basis weight in the range between 150 and 300 gsm, such as between 200 and 250 gsm. For example, the one or more fabric panels may comprise a textile fabric having a basis weight in the range between 150 and 300 gsm, such as between 200 and 250 gsm. Such a fabric could be a knitted textile fabric, such as a weft knit textile fabric, e.g. a weft knit single jersey as set out in the above.

The outer cover may comprise a material that is elastic in at least one direction, preferably in two directions. For example, the one or more fabric panels may comprise a material that is elastic in at least one direction, preferably in two directions. Such a material could be e.g. a knitted fabric as set out in the above.

For example, the outer cover may comprise a textile fabric comprising polyester.

As such, the one or more of the fabric panels of the garment portion may comprise a textile fabric comprising polyester. For example, the majority of the area of the garment portion may comprise a textile fabric comprising polyester, such as at least 60% of the area, or at least 80% or at least 90% or the area.

For example, the one or more fabric panels may comprise a textile fabric comprising a mixture of polyester and elastane. For example, the one or more fabric panels may comprise a textile fabric comprising no less than 70% polyester, such as no less than 80% polyester. For example, one or more fabric panels may comprise a textile fabric comprising 70 to 90% polyester, and 30 to 10% elastane. For example, the one or more fabric panels may comprise a fabric comprising 80% polyester and 20% elastane.

Polyester materials have been found to have advantageous properties throughout use and washing cycles, for example in relation to shrinking, and to retaining its shape and stretch.

For example, the outer cover may comprise a textile fabric comprising recycled material. As such, one or more of the fabric panel or panels of the garment portion may comprise a textile fabric comprising recycled material. For example, the majority of the area of the garment portion may comprise a textile fabric comprising recycled material, such as at least 60% of the area, or at least 80% or at least 90% or the area.

For example, the amount of recycled material in the textile fabric may be at least 20%, such as at least 50%, or at least 70%.

As will described in the below, the outer cover disclosed herein may comprise a plurality of parts being joined to form the front portion, the back portion, and the crotch portion of the garment portion. Use of more than one part may, as will be described in the below, provide for several options for controlling the shape of the outer cover and/or other properties, such as elasticity, of the outer cover.

The different parts may be joined by any suitable joining members. For example, the joining members could be selected considering the requirements for e.g. washability and/or breathability of the outer cover.

For example, the different parts may be joined by adhesive joining members, such as adhesive tape.

For example, the different parts may be joined by mechanical joining members such as stiches or seams.

The different parts may be permanently joined, i.e. intended to be joined throughout the life cycle of the outer cover.

Optionally, different parts of the outer cove may be made by different fabrics. As such, different properties of different fabrics may be used to impart different functions and/or aesthetics to the garment portion and to the outer cover.

In another option, when the different parts of the outer cover may be made by the same fabric. As such, the different parts will have similar properties, i.e. liquid permeability and/or breathability, and similar behaviour when subject to use, such as washing and wearing. This may be advantageous for the durability of the reusable outer cover. Moreover, using the same fabric material for the different portions may provide for efficient use of fabric material during manufacture, reducing fabric material waste.

As mentioned in the above, the absorbency of the absorbent article to be formed by the outer cover, when an absorbent insert is attached thereto, is primarily to be provided by the absorbent insert alone.

For example, the crotch portion of the outer cover may be essentially non-absorbing, implying that the crotch portion is not intended to absorb any bodily discharges to a significant degree.

For example, the entire outer cover may be essentially non-absorbing.

With essentially non-absorbing is meant herein that the outer cover is not intended to absorb and retain fluids to a significant degree.

To this end, the one or more different portions of the outer cover may consist of one or more materials, each material being essentially non-absorbing.

### General Description of Variants of the Outer Cover as Disclosed Herein

The outer cover 100 has, as in the depicted variant, a front portion Sf to be directed towards a front of a wearer when the outer cover is in use, a back portion Sb to be directed towards a back of a wearer when the outer cover is in use, and a crotch portion Sc extending between the front portion Sf and the back portion Sb. The outer cover 100 comprises a front edge 101 at the front portion Sf of the outer cover, and a back edge 102 at the back portion Sb of the outer cover.

The outer cover 100 further comprises a first side edge 103 and a second side edge 104 respectively extending between the front and back edges 101, 102 of the outer cover 100.

The outer cover 100 extends along a central longitudinal axis Y from the front edge 101 at the front portion Sf of the outer cover to the back edge 102 at the back portion Sb, and the outer cover 100 extends between the first side edge 103 and the second side edge 104 along a central transversal axis X, which is perpendicular to the longitudinal axis Y. Further, a thickness axis Z is defined perpendicular to both the longitudinal and transversal axes X, Y.

As seen in FIGS. 1a-1b, an inward direction Zi is defined along the thickness axis Z being the direction towards a wearer when the outer cover 100 is worn, and an outward direction Zo is defined along the thickness axis Z being the direction away from the wearer when the outer cover is worn.

The crotch portion Sc of the outer cover defines a minimum crotch transversal width Wmin of the outer cover, and the central longitudinal axis Y extends centrally through the minimum crotch transversal width Wmin. A central outer cover length L is measured between the front edge 101 and the back edge 102 at the central longitudinal axis Y, Hence, the central outer cover length L is the length between the front edge 101 and the back edge 102 along the longitudinal axis Y, and at the longitudinal axis Y, i.e. at centrally through the minimum crotch transversal width Wmin.

At least one out of the back portion Sb or the front portion Sf of the outer cover comprises a pair of side portions 110, 111 extending transversally on each side of the central longitudinal axis Y. Each of the side portions 110, 111 of the outer cover comprises one or more waist attachment elements 115, 116 to attach the outer cover around the waist of a wearer.

According to examples, and as in the outer cover of the Figures, at least the back portion Sb comprises a pair of side portions 110, 111.

According to examples, such as the outer cover illustrated in the Figures, the side portions 110, 111 are attachable to the front portion Sf of the outer cover, to fasten the outer cover around the waist of a wearer. For example, and as in the illustrated outer cover, the outward facing side of the front portion may be provided with one or more complementary attachment elements 117, 118 (see FIG. 2) to which the one or more waist attachment elements 115, 116 of the side portions 110, 111 are detachably attachable to close the outer cover around the waist of a wearer.

In other, non-illustrated examples, the outer cover may also comprise a pair of front side portions extending transversally on each side of the longitudinal axis Y in the front portion Sf of the outer cover, and the back side portions may be attachable to the front side portions to fasten the outer cover around to the waist of the wearer.

In yet other, non-illustrated examples, the back side portions are attachable to each other to form a closed position i.e. a closed belt around the waist of the wearer, and the front portion is attachable to the side portions when in the closed position.

In accordance with the present disclosure, and as illustrated in the outer cover example of the figures, the outer cover 100 is further defined as follows:

The outer cover 100 comprises a chassis portion 150 extending along the longitudinal axis y from a front chassis edge 101c forming part of the front edge 101 of the outer cover to a back chassis edge 102c forming part of the back edge 102 of the outer cover. The chassis portion 150 comprises a first chassis side edge 103c and a second chassis side edge 104c respectively extending between the chassis front and back edges 101c, 102c of the chassis portion 150, on opposite sides with respect to the longitudinal axis Y.

As mentioned in the above, at least one out of the back half Sb and the front half Sf of the outer cover comprises a first and a second side portion 110, 111. The first and second side portions 110, 111 extend transversally on opposite sides of the chassis portion 150 with respect to the longitudinal axis Y, wherein each of the side portions 110, 111 of the outer cover comprises one or more waist attachment elements 115, 116 to attach the outer cover around the waist of a wearer during use.

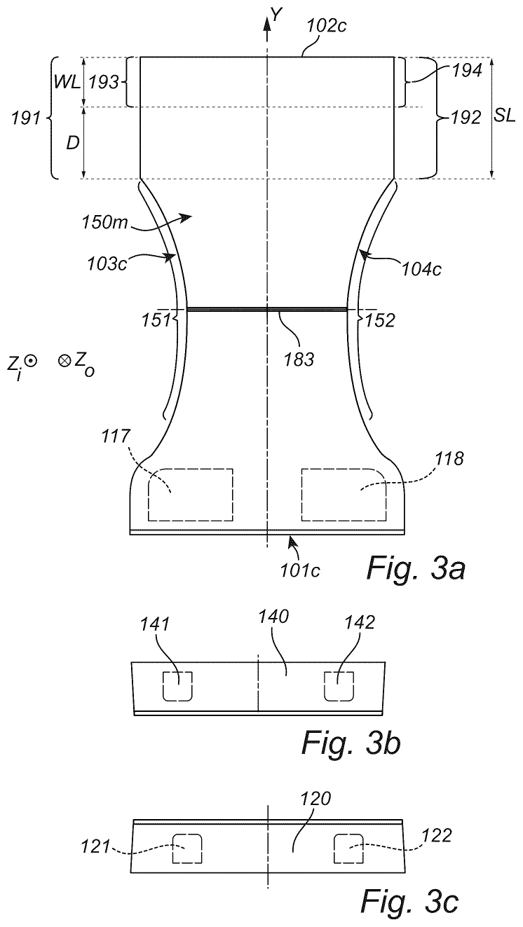

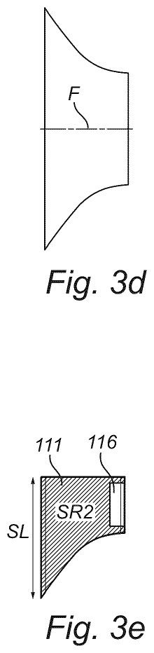

The first side portion 110 is permanently attached to the chassis portion 150 along a first side attachment segment 191 of the first chassis side edge 103c, and the second side portion 111 is permanently attached to the chassis portion 150 along a second side attachment segment 192 of the second chassis side edge 104c. The first and second side attachment segments 191, 192 define a side portion length SL from the longitudinal ends of each segment closest to the central transversal axis X at the respective first and second chassis side edges 103c, 104c and to the adjacent back edge 102 or front edge 101 of the outer cover. (See FIGS. 1a-1b and FIG. 3a.)

The chassis portion 150 comprises a main member 150m and a flap member 140. The main member 150m forms the front or back chassis edge 101c, 102c, the first chassis side edge 103c, and the second chassis side edge 104c in the front or back half. For example, the main member 150m may extend over the entire chassis portion 150 of the outer cover 200, i.e. over both the front and back half, and form the front and back chassis edges 101c, 102c and the first and second chassis side edges 103c, 104c. (See FIG. 3a—main member, and FIG. 3b—flap member.)

The flap member 140 is arranged with an outward facing side of the flap member 140 facing an inward facing side of the main member 150m so as to form a pocket between the outward facing side of the flap member 140 and the inward facing side of the main member 150m. The pocket is intended to receive a back end portion or a front end portion, respectively, of an absorbent insert to be detachably attached to the outer cover to form an absorbent article.

The flap member 140 is permanently attached to the main member 150m along a first flap attachment segment 193 of the first chassis side edge 103c and along a second flap attachment segment 194 of the second chassis side edge 104c (See FIG. 3a.). The first and second flap attachment segments 193, 194 define a waist region length WL from the longitudinal ends of each segment closest to the central transversal axis X and to the back edge 102 of the back half, or the front edge 101 of the front half, of the outer cover.

It is to be understood that the side portion length SL, the waist region length WL, and the longitudinal distance D are all measured parallel to the longitudinal axis Y.

The side portion length SL extends by a longitudinal distance D beyond the waist region length WL in the direction towards the transversal axis X.

When the outer cover is in use, the first and second side portions 110, 111 will be pulled in a transversal direction so as to fasten the outer cover around the waist of the wearer. The opposite transversal forces applied to the first and second side portions 110, 111 will be transferred to the chassis portion 150 since the first and second side portions 110, 111 are permanently attached to the chassis portion 150 along the first and second side attachment segments 191, 192 of the first and second chassis side edges 103c, 104c.

By virtue of the side portion lengths SL extending beyond the waist region length WL in the direction towards the transversal axis X by the distance D, it is ensured that the transversal forces applied to the first and second side portions 110, 111 when the outer cover is in use will be transferred not only to the part of the chassis portion 150 including the waist region part of the chassis portion 150 where the flap member 140 is attached, but also to a region of the chassis portion 150 extending beyond the waist region towards the transversal axis X, i.e. to a region of the chassis portion 150 not including the flap member 140. Thus, the first and second side portions 110, 111 will ensure that the part of the main member 150m extending longitudinally along the distance D is stretched out in the transversal directions when the outer cover is in use. Thus, it may be ensured that the main member 150 covers the wearer and does not unintentionally fold or wrinkle towards the longitudinal axis Y. This may be of particular importance in the back half of the outer cover, since the back portion of the chassis 150 may be shaped to cover the buttocks and lower back region of the wearer, e.g. to have a relatively large width. In an absorbent article comprising a reusable outer cover and a detachable insert, it is advantageous if the size of the insert may be reduced, in particular if the insert is to be disposable. Thus, the insert may be optimized so as to perform the necessary absorbing function. The outer cover may be optimized so as to hold the insert in a correct position visavi the wearer during use, but also to ensure that the absorbent article sufficiently covers the wearer. This is of importance for the comfort, fit, and dignity of the wearer. Thus, by ensuring that the side portions 110, 111 cooperates with the chassis portion as explained in the above, a better coverage and fit may be provided by the outer cover, by still performing the function of detachably holding the insert in correct position visavi the wearer.

By an item being “permanently attached along a segment of an edge” is to be understood that the attachment is made at or along the edge. For example, the attachment could be made with a distance to the very edge, as may be required by the type of joint used to perform the attachment. For example, the attachment could be made by a seam running along the segment of an edge with a seam allowance between the edge and the seam.

According to examples, such as the outer cover of FIGS. 1a-1b, the first and second side attachment segments 191, 192 extend essentially parallel to the longitudinal axis Y (see FIG. 3a). When the first and second side attachment segments 191, 192 extend essentially parallel to the longitudinal axis Y it may be ensured that the opposite transversal forces applied to the first and second side portion 110, 111 when the outer cover is in use is evenly transferred to the chassis portion 150.

According to examples, such as the outer cover of FIGS. 1a-1b, the first and second flap attachment segments 193, 194 extend essentially parallel to the longitudinal axis Y (see FIG. 3a). When the first and second flap attachment segments 193, 194 extend essentially parallel to the longitudinal axis Y it may be ensured that the opposite transversal forces applied to the first and second side portions 110, 111 when the outer cover is in use are evenly transferred to the flap member 140, contributing to stretching out the flap member 140 in the transversal directions.

According to examples, such as the outer cover of FIGS. 1a-1b, the first and second side attachment segments 191, 192 and the first and second flap attachment segments 193, 194 extend essentially parallel to the longitudinal axis Y. This ensures the above-mentioned advantages relating to force distribution, and moreover provides for efficient possibilities for manufacturing the outer cover.

According to examples, such as the outer cover of FIGS. 1a-1b, the outer cover comprises
a first joint assembly 181 comprising one or more joints connecting the first side portion 110 to the chassis portion 150 along the first side attachment segment 191, and connecting the flap member 140 to the main member 150m along the first flap attachment segment 193 and
a second joint assembly 182 comprising one or more joints connecting the second side portion 111 to the chassis portion 150 along the second side attachment segment 192, and connecting the flap member 140 to the main member 150m along the second flap attachment segment 194.

A joint assembly could comprise a plurality of joints, for example, the joint assembly could comprise two joints. For example, the first joint assembly could comprise one joint connecting the first side portion 110 to the chassis portion 150 along the first side attachment segment 191, and another joint connecting the flap member 140 to the main member 150m along the first flap attachment segment 193. Similarly, the second joint assembly could comprise one joint connecting the second side portion 111 to the chassis portion 150 along the second side attachment segment 192, and another joint connecting the flap member 140 to the main member 150m along the second flap attachment segment 194.

To achieve the desired transmission of transversal forces to the chassis portion 150 when the outer cover comprises a first and a second joint assembly 181, 182 as described in the above, a transversal spacing between any joints in each joint assembly 181, 182 may be kept relatively narrow. For example, a maximum transversal spacing between any joints in each joint assembly 181, 182 may be less than 2 cm, preferably less than 1 cm. Also, the joints may be arranged so as to completely or partly overlap each other as seen along the thickness axis X, i.e. such that there is no transversal spacing between the joints in each joint assembly 181, 182.

According to examples, such as the outer cover of FIGS. 1a-1b, the outer cover comprises a first joint 181 connecting the first side portion 110 to the chassis portion 150 along the first side attachment segment 191, and connecting the flap member 140 to the main member 150m along the first flap attachment segment 193. Further, the outer cover comprises a second joint 182 connecting the second side portion 111 to the chassis portion 150 along the second side attachment segment 192, and connecting the flap member 140 to the main member 150m along the second flap attachment segment 194.

In other words, the first side portion 110 and the flap member 140 are both permanently attached to the chassis portion 150 by a first joint 181, and the second side portion 111 and the flap member 140 are both permanently attached to the chassis portion 150 by a second joint 182.

This provides for efficient force distribution from the first and second side portions 110, 111 to the chassis portion 150, such that both the waist region having the flap member 140, and the main member region extending along the above-mentioned first distance D may be stretched out in transversal directions. Moreover, this provides for efficient manufacturing of the outer cover.

According to examples, each of the joint or joints, such as each of the first and second joints 181, 182, is a stitched seam, a welded seam, an adhesive seam, an adhesive tape or combinations thereof.

For example, such as in the outer cover of FIGS. 1a-1b, each of the joint or joints 181, 182 is in the form of a stitched seam.

The joint or joints preferably have a relatively low stretchability in the direction cross the length extension of the joint. As such, the joint or joints are stable to transmit forces in the cross direction of the joint, and so, when the joint or joints are arranged to extend primarily in the longitudinal direction of the outer cover, the joint or joints are stable to transmit forces along the transversal direction of the outer cover. The types of joints mentioned in the above typically do have a relatively low stretchability in the direction cross the length extension of the joint. The joint or joint may optionally have a relatively higher stretchability in the direction along the length extension of the joint.

The joint or joints could be intermittent, i.e. formed by separate stitches or adhesive connections.

However, it is preferred that the joint or joints are continuous. In this sense, a stitched seam is regarded as “continuous.”

According to examples, such as the outer cover of FIGS. 1a-1b, the joint or joints 181, 182 extend essentially parallel to the longitudinal axis Y.

According to examples, and as in the outer cover of FIGS. 1a-1b, the longitudinal distance D is no less than 0.50× the waist region length WL. For example, the longitudinal distance D may be no less than 0.75× the waist region length WL or no less than 0.90× the waist region length WL. The longitudinal distance D may be no more than 2.00× the waist region length WL such as no more than 1.50× the waist region length WL. According to examples, and as in the outer cover of FIGS. 1a-1b, the longitudinal distance D is from 0.50× the waist region length WL to 2.00× the waist region length, for example from 0.75× the waist region length WL to 2.00× the waist region length WL. According to examples, such as in the illustrated outer cover, the longitudinal distance D is from 0.90× the waist region length to 1.50× the waist region length. In the illustrated outer cover, the longitudinal distance D is approximately 1.0× the waist region length WL.

According to examples, and as in the outer cover of FIGS. 1a-1b, the longitudinal distance D is at least 5 cm, such as at least 10 cm. According to examples, and as in the outer cover of FIGS. 1a-1b, the longitudinal distance D is no more than 20 cm. For example, and as in the outer cover of FIGS. 1a-1b, the longitudinal distance D may be from 5 to 20 cm, such as from 10 to 20 cm.

According to examples, and as in the outer cover of FIGS. 1a-1b, the side portion length SL is from 20% to 30% of a central outer cover length L of the outer cover, preferably from 22% to 27%, most preferred about 25% of the central outer cover length L of the outer cover.

According to examples, and as in the outer cover of FIGS. 1a-1b, the waist region length WL is less than 15% of a central outer cover length L of the outer cover, preferably less than 10% of a central outer cover length L of the outer cover.

### Cloth Percentual Stretch of Different Regions

According to examples, a reusable outer cover 100 according the first variant of this disclosure, may be defined in terms of the Cloth Percentual Stretch in different regions of the outer cover 100, wherein the Cloth Percentual Stretch is measured in accordance with the Test Method described herein.

The Cloth Percentual Stretch in a region of the outer cover is a property of the cloth material making up the region of the outer cover. Thus, the Cloth Percentual stretch is measured over a cloth region lacking joints and without any additional members (such as insert fastener members being attached to the cloth.

With a “region” of the outer cover is meant a region as seen in the plane spanned by the transversal axis X and the longitudinal axis Y. Thus, a region may comprise one or more layers being superposed in the thickness direction X. For example, one layer from the main member 150m of the chassis portion and one layer from the flap member 140.

For example, the main member 150m has a first transversal Cloth Percentual Stretch in a region extending between the first side attachment segment 191 and the second side attachment segment 192, and along the side portion length SL. The flap member 140 and the main member 150m together have a second transversal Cloth Percentual Stretch in a waist region extending between the first flap attachment segment 193 and the second flap attachment segment 194 and along the waist region length WL. The second transversal Cloth Percentual Stretch is less than the first transversal Cloth Percentual Stretch.

Moreover, in a second, alternative variant of the disclosure, there is provided a reusable outer cover 100 for forming an absorbent article 1 wherein an absorbent insert is adapted to be detachably attached to the outer cover. The outer cover 100 has a front portion Sf to be directed towards a front of a wearer, a back portion Sb to be directed towards a back of a wearer, and a crotch portion Sc extending between the front portion Sf and the back portion Sb,
the outer cover 100 extending along a longitudinal central axis Y, and along a transversal central axis X, which is perpendicular to the longitudinal axis Y, the transversal central axis X dividing the outer cover 100 into a front half and a back half, and a thickness axis Z is defined perpendicular to both the longitudinal and transversal axes X, Y,
the outer cover 100 comprising a chassis portion 150 extending along the longitudinal axis y from a front chassis edge 101c at the front edge 101 of the outer cover to a back chassis edge 102c at the back edge 102 of the outer cover,
the chassis portion 150 comprising a first chassis side edge 103c and a second chassis side edge 104c respectively extending between the chassis front and back edges 101c, 102c of the chassis portion 150, on opposite sides with respect to the longitudinal axis Y, wherein,
in at least one out of the back half Sb and the front half Sf of the outer cover,
the outer cover comprises
a first and a second side portion 110, 111 extending transversally on opposite sides of the chassis portion 150 with respect to the longitudinal axis Y to fasten the outer cover around the waist of a wearer, wherein each of the side portions 110, 111 of the outer cover comprises one or more waist attachment elements 115, 116 to attach the outer cover around the waist of a wearer,
the first side portion 110 is permanently attached to the chassis portion 150 along a first side attachment segment 191 of the first chassis side edge 103c, and the second side portion 111 is permanently attached to the chassis portion 150 along a second side attachment segment 192 of the second chassis side edge 104c, the first and second side attachment segments 191, 192 defining a side portion length SL from the longitudinal ends of each segment closest to the central transversal axis X and to the adjacent back edge 102 or front edge 101 of the outer cover,
AND
The chassis portion 150 comprises a waist region WR extending transversally between the first and second side attachment segments 191, 192, and
Longitudinally from the front or back edge 101, 192 of the outer cover and towards the central longitudinal axis X along a waist region length WL,
And the chassis portion 150 comprising an intermediate region IR extending transversally between the first and second side attachment segments 191, 192, and longitudinally from the waist region towards the central longitudinal axis X along a first distance D,
wherein
the intermediate region IR has a first transversal Cloth Percentual Stretch, and the waist region WR has a second transversal Cloth Percentual Stretch, and the second transversal Cloth Percentual Stretch is less than the first transversal Cloth Percentual Stretch. (See FIG. 1b)

This second variant of the disclosure may be combined with the various features and advantages of the first variant of the disclosure as set out in the above.

For both variants of the disclosure, further advantageous features relating to the Cloth Percentual Stretch properties are exemplified in the following.

According to examples, such as in the depicted outer cover, the second transversal Cloth Percentual Stretch is from 30 to 70% of the first transversal Cloth Percentual Stretch, such as from 40 to 60% of the first transversal Cloth Percentual Stretch, such that from 45 to 55% of the first transversal Cloth Percentual Stretch.

For example, the second transversal Cloth Percentual Stretch may be about 50% of the first transversal Cloth Percentual Stretch.

According to examples, such as in the depicted outer cover, each one out of the first and the second side portions 110, 111 forms a side region SR1, SR2, wherein each side region SR1, SR2 has a side portion transversal Cloth Percentual Stretch, the side portion transversal Cloth Percentual Stretch being less than the first transversal Cloth Percentual Stretch.

According to examples, such as in the depicted outer cover, the side portion transversal Cloth Percentual Stretch is from 30 to 70% of the first transversal Cloth Percentual Stretch, such as from 40 to 60% of the first transversal Cloth Percentual Stretch, such that from 45 to 55% of the first transversal Cloth Percentual Stretch.

For example, the side portion transversal Cloth Percentual Stretch may be about 50% of the first transversal Cloth Percentual Stretch

### Gathered Edge Segments

According to examples, such as the outer cover of FIGS. 1a-1b, the outer cover of the first or the second variant of the disclosure further comprises a first elastic edge element 161 and a second elastic edge element 162. The first elastic edge element 161 is attached along at least a part of the first chassis side edge 103c and the first elastic edge element 161 is adapted to
gather a first gathered edge segment 151 of the first chassis side edge 103c when the first elastic edge element 161 is in a relaxed condition and the first gathered edge segment 151 is gathered with a gathered extension and;
stretch out the first gathered edge segment 151 of the first chassis side edge 103c when the first elastic element 161 is in an outstretched condition and the first gathered edge segment 151 is flat with an ungathered extension.

The second elastic edge element 162 is attached along at least a part of the second side edge 104 and the second elastic edge element 162 is adapted to
gather a second gathered edge segment 152 of the second chassis side edge 104c when the second elastic edge element 162 is in a relaxed condition and the second gathered edge segment 152 is gathered with a gathered extension and
   stretch out the second gathered edge segment 152 of the second chassis side edge 104c when the second elastic element 162 is in an outstretched condition and the second gathered edge segment 152 is flat with an ungathered extension.

Accordingly, a first and a second gathered edge segment 151, 152 is formed along the first side edge 103 and the second side edge 104, respectively, of the outer cover 100. The gathered edges segments 151, 152 may thus promote that the leg openings formed by the outer cover 100 when in use adapt to the legs of the wearer and fit towards the legs of the wearer. As such, the first and second gathered edge segments 151, 152 promote a better fit of the outer cover in the region around the legs of the wearer. In addition, the first and second gathered edge segments 151, 152 may assist in holding the detachable absorbent insert 200, when attached to the outer cover 200, in a correct position visavi the wearer during use.

The first and second elastic edge elements 161, 162 may be any type of elastic element, such as an elastic thread, ribbon or band, which is applied at or adjacent the first or second chassis side edge 103c, 104c. For example, and as in the outer cover of the Figures, each of the first and second elastic edge elements 161, 162 may be an elastic edging. Such an elastic edging may be folded around a first or second edge of one or more material panels forming the outer cover, such that the elastic edging forms at least part of the first side edge 103 or the second side edge 104 of the outer cover.

The first and second elastic edge elements 161, 162 are applied at least along the first and second gathered edge segments 151, 152, respectively. However, the first and second elastic edge elements 161, 162 may extend beyond the first and second gathered edge segments 151, 152 along the first or second side edge 103, 104, respectively, in the direction towards the rear edge 102 and/or the front edge 101.

For example, and as in the outer cover of FIGS. 1a-1b, the elastic edge elements 161, 162 in the form of elastic edgings may extend along the first side edge 103 and the second side edge 104 from the front end 101 of the outer cover to the one or more waist attachment elements 115, 116 of the first side portion 110 and the second side portion 111, respectively.

According to examples, such as the outer cover of FIGS. 1a-1b, a ratio of the ungathered extension of the first and second gathered edge segments 151, 152 to the gathered extension of the respective first and second gathered edge segments 151, 152 is in the range from 2.5:1 to 3.0:1.

The ungathered extension is to be measured when the outer cover is laid out as flat as possible and with the elastic edge element being stretched out such that the gathered edge segments are flat, without ruffles, but not stretched. The measurement is made along the first or second side edge 103, 104, respectively (i.e. not parallel to longitudinal axis Y, but following the curve of the side edge 203, 204.

The gathered extension is to be measured when the cover is laid out as flat as possible and with the elastic edge element being active and relaxed, i.e. not stretched out but in a resting position. The measurement is again made along the first or second side edge 103, 104, respectively, when in its gathered state.

According to examples, and as in the outer cover of FIGS. 1a-1b, the first and second side attachment segments 191, 192 are ungathered.

When the first and second side attachment segments 191, 192 extend beyond the first and second flap attachment segments 193, 194 by a first distance D as proposed herein, the first and second side attachment segments 191, 192 may promote stretching of the first and second elastic edge elements 161, 162 when the article is in use. I.e. the transversal opposite forces submitted to the first side portion 110 and to the second side portion 111 when the article is attached to a wearer may be transferred to the chassis portion so as to stretch the first and second elastic edge elements 161, 162 and the corresponding first and second gathered edge segments 151, 152. Thus, activation of the elastic edge elements may be ensured to the benefit of the fit of the outer cover.

According to examples (not illustrated), the first gathered edge segment 151 and the first side edge segment 191 may be spaced by a second longitudinal distance, and the second gathered edge segment 152 and the second side edge segment 192 may be spaced by the second longitudinal distance. The second longitudinal distance may be no more than 3 cm, preferably no more than 2 cm.

By the second longitudinal distance being relatively short, the stretching of the first and second elastic edge elements 161, 162 as described in the above may be promoted.

According to examples, and as in the outer cover of FIGS. 1a-1b, the first gathered edge segment 151 follows directly from the first side attachment segment 191, and the second gathered edge segment 152 follows directly from the second side attachment segment 192. (In other words, any second longitudinal distance between the first/second gathered edge segment and the first/second side attachment segment is zero.)

By the first and second gathered edge segments 151, 152 following substantially directly from the first side attachment segment 191 and the second side attachment segment 192, the opposite transversal forces to which the first and second side portions 110, 111 are subject when the article is applied to a wearer may be particularly efficiently transferred to stretch out the first and second elastic edge elements 161, 162.

According to examples, and as in the outer cover of FIGS. 1a-1b, the first joint assembly or first joint 181 overlaps with the first elastic edge element 161, and the second joint assembly 182 or second joint 182 overlaps with the second elastic edge element 162, as seen in the thickness direction Z.

The first and second gathered edge segments 151, 152 each extends towards the transversal axis X. According to examples, and as in the outer cover of FIGS. 1a-1b, each gathered edge segment 151, 152 extends over the transversal axis X, towards the opposite of the back portion Sb and the front portion Sf of the outer cover.

According to examples, such as the outer cover of FIGS. 1a-1b, each of the first side edge 103 and second side edge 104 of the outer cover is free from gathered edge segments in the longitudinal part of the outer cover 100 which extends along the side portion length SL. Thus, the side portions 110, 111 are free from gathered edge segments.

When the outer cover, as in the illustrated example, comprises first and second gathered edge segments 151, 152 which each extends from a position in the front portion of the outer cover, and substantially to the crotchmost end of the side portion length SL, and the outer cover is free from gathered edge segments in the longitudinal part of the outer cover extending along the side portion length SL to the back edge 102, the gathered edge segments 151, 152 may be particularly well suited to provide for adaption of the outer cover to the wearer's legs, while the outer cover may extend in a transversal direction so as to provide sufficient cover for the wearer's buttocks and lower back region.

Moreover, according to examples, such as the outer cover of FIGS. 1a-1b, each of the first side edge 103 and the second side edge 104 may be free from gathered edge segments in the front region of the outer cover, for example, at least in a longitudinal portion of the outer cover extending from the front edge 101 and so as to comprise the front insert fastener members 121, 122.

Further, the front edge 101 and/or the back edge 102 may be free from gathered edge segments, preferably both the front edge 101 and the back edge 102 are free from gathered edge segments. This is because for the improved fit desired herein, gathered segments along the front edge and the back edge are generally undesired. Still, in examples, as in the outer cover of FIGS. 1a-1b, the entire or part of the front edge 101 and/or the back edge 102 may be provided with an elastic edging 163 for forming a soft and sustainable front or rear edge 101, 102, respectively.

According to examples, such as the outer cover of FIGS. 1a-1b, a maximum width between the first and second side edge 103, 104 along the first and second gathered edge segments 151, 152 is greater than a transversal insert fastener width oWf spanned by the back insert fastener areas 141, 142, as will be described in more detail in the below.

### Flap Members of the Outer Cover

The flap member 140 may extend longitudinally from a position adjacent the front edge 101 or the back edge 102 respectively, and towards the transversal axis X.

The flap member may for example have a longitudinal length being no more than 20% of the outer cover length L, such as no more than 15% of the outer cover length L, or no more than 10% of outer cover length. As set out in the above, a central outer cover length L is measured between the front edge 101 and the back edge 102 at the central longitudinal axis Y. Hence, the central outer cover length L is the length between the front edge 101 and the back edge 102 along the longitudinal axis Y, and at the longitudinal axis Y, i.e. at centrally through the minimum crotch transversal width Wmin.

The flap member being relatively short as compared to the longitudinal length of the outer cover, implies that the flap member will leave a substantial portion of the inward facing side of the main member 150m uncovered, as seen from the inward direction of the outer cover. Hence, in use, when an absorbent insert is attached to the outer cover, and arranged with a front or back end portion in the pocket formed between the flap member 140 and the main member 150m, the absorbent insert will form the inward facing surface of the absorbent article over this substantial portion. Thus, the flap member 140 will leave a substantial portion of the inward facing surface of the insert free to receive bodily discharge from the wearer.

The flap member 140 may be formed by a separate piece of material, such as a fabric panel, being connected to the main member 150m via one or more joints as described in the above. For example, the flap member 140 may have a front or back edge directed towards the front chassis edge 101c or the back chassis edge 102c of the chassis portion, a free edge directed towards the transversal axis X, and a first and a second side edge connecting the front or back edge and the free edge.

The first and second side edges are transversally spaced and extend in a longitudinal direction, for example the first and second side edges may extend essentially parallel to the longitudinal direction of the outer cover.

The free edge is free from attachment to the main member 150m along at least a majority of its width, such that a front or back end of an insert is insertable into the pocket formed between the flap member 140 and the main member 150m, the pocket being open towards the transversal axis X. The free edge may extend essentially parallel to the transversal axis X. Optionally, the free edge may have a slightly curved shape.

The first side edge of the flap member 140 may be permanently connected to the main member 150m along the first flap attachment segment 193, and the second side edge of the flap member 140 may be permanently connected to the main member 150m along the second flap attachment segment 194, as described in the above.

When the flap member 140 is formed by a separate piece of material, the front or back edge of the flap member 140 may be connected to the main member 150m at or adjacent the front or back edge of the chassis portion 150 via one or more joints.

Alternatively, the flap member 140 may be formed from a piece of material which also forms the main member 150m. In this case, the flap member 140 may be folded towards the main member 150m along a fold line, such that the fold line forms a front or back end of the flap member 140, and optionally also the front or back edge 101c, 102c of the chassis portion 150. Also in such variants, the first and second side edges of the flap member may be permanently connected to the main member 150m as described in the above, and the flap member may comprise a free edge as described in the above.

The flap member may have a transversal width being essentially the same as the transversal width of the main member 150m between the first and second flap attachment segments 193, 194.

For example, the transversal width between the first and second flap attachment segments 193, 194, and/or the transversal width between the first and second side attachment segments 191, 192 may be at least 120% of the minimum transversal crotch width Wmin of the outer cover. Optionally, the transversal widths may be at least 130% of the minimum crotch width Wmin of the outer cover, such as at least 140% of the minimum crotch width Wmin of the outer cover.

According to examples, and as in the illustrated outer cover, the free edge of the flap member 140 may have a transversal width being slightly less than the transversal width between the first and second flap attachment segments 193, 194. This ensures that at least the free edge of the flap member 140 is stretched such that it does not risk folding “up” when the outer cover is in use.

### Insert Fastener Areas

According to examples, and as in the outer cover of FIGS. 1a-1b, a waist region WR of the chassis portion is defined transversally between the first and second flap attachment segments 193, 194, the waist region having the waist region length WL. Further, the waist region of the chassis portion 150 may comprise one or more insert fastener areas 141, 142 for detachably fastening an insert to the outer cover. Preferably the insert fastener areas 141, 142 comprises at least two insert fastener areas 141, 142 being transversally spaced and located on each transversal side of the central longitudinal axis Y.

According to examples, and as in the outer cover of FIGS. 1a-1b, at least one out of the outward facing side of the flap member 140 and the inward facing side of the main member 150m forming the pocket comprises one or more insert fastener areas 141, 142 for detachably fastening an insert to the outer cover.

According to examples, such as in the example of FIGS. 1a-1b, the outward facing side of the flap member 140 comprises one or more insert fastener areas 141, 142 for detachably fastening an insert to the outer cover. With the one or more insert fastener areas 141, 142 being positioned on the flap member, any undesired aesthetical effects on the outer cover, resulting from the insert fastener area(s), (such as for example seams attaching an insert fastener member to outer cover), will appear on the flap member only and not be visible from the outside of the garment portion, when the outer cover is worn. The outward facing surface of the garment portion is intended to be the outward facing surface of the complete absorbent article, and hence it will be directed towards a wearer's regular garments when the outer cover is in use.

Further, that the one or more insert fastener areas is/are positioned on the outward facing surface of the flap member, which surface is spaced from the garment portion, implies that, when the outer cover is in use, the movement of the garment portion is less restricted by the insert fastener area and thus the insert. For example, any area of the garment portion over which the flap member extends will be able to follow the general movement of the garment portion, and so, the outward appearance of the garment portion will be more underwear-like, without visible tension in the garment portion resulting from the attachment of the insert to the outer cover.

Similarly, the insert fastener areas, and thus the position of the insert, will be less prone to being dislocated by e.g. the garment portion being subject to tearing or friction, resulting from the wearer's movements.

The one or more insert fastener areas 141, 142 positioned on the outward facing inner side of the flap member 140 may be the only insert fastener elements for detachably attaching an insert to the outer cover in the front or back portion of the outer cover.

Preferably, as in the illustrated outer cover, and as will be described in more detail below, each of the front portion Sf and the back portion Sb of the outer cover comprises a front/back set of one or more insert fastener areas 141, 142; 121, 122 positioned on the outward facing inner side of a back flap member 140 or a front flap member 120, respectively, wherein the front and back sets of insert fastener areas are the only insert fastener elements for detachably attaching an insert to the outer cover, of the outer cover.

For example, the area of the inward facing surface of the main member 150m over which the flap member 140 extends (when in the use position may be free from insert fastener areas. Optionally, the entire main member 150m may be free from insert fastener areas. As such, the only attachment between the outer cover and an insert may be made via the flap members 120, 140 comprising the sets of insert fastener areas 121, 122; 141, 142, which may be configured for detachable fastening towards the inward facing side of an absorbent insert 200.

Preferably, the one or more insert fastener areas 141, 142 may be entirely contained within flap member 140, e.g. be entirely contained within the one or more fabric panels of the flap member(s), preventing any direct contact between the fastener areas and the wearer's skin. For example, the flap member(s) may comprise one or more fabric panels and one or more insert fastener areas being contained within the one or more fabric panels. By “entirely contained” is meant that the one or more insert fastener areas do not extend beyond, e.g. does not protrude from, the one or more flap members/fabric panels.

For example, the insert fastener areas may be provided by insert fastener members attached to the one or more fabric panels, as will be further described in the below.

For example, each insert fastener member may comprise at least one insert fastener area.

Each insert fastener member may be attached to the chassis portion 150 of the outer cover in the waist region WR.

According to examples, as in the outer cover of FIGS. 1a-1b, the insert fastener members may be attached to the outward facing side of the flap member 140.

For example, an insert fastener member may comprise a sheet formed base having a length in the longitudinal axis and a width in the transversal axis, the sheet formed base comprising one or more areas of surface fastening material, i.e. of the first or the second surface fastening material.

Preferably, each insert fastener member comprises one single insert fastener area comprising a surface fastening material, i.e. the first or the second surface fastening material. This may be advantageous to reduce the amount of material added to the insert, and/or to reduce any additional stiffness implied by the presence of an insert fastener member on the outer cover.

For example, an insert fastener member may be a piece of a hook material or of a loop material. A hook/loop material may be a material comprising a sheet-formed base having a surface upon which the hooks/loops are arranged. For example, an insert fastener member may be formed from a piece of hook/loop material cut from a band-shaped hook material or loop material. In order to ensure that the outer cover is washable, the surface fastening materials of the insert fastener areas, e.g. the insert fastener members, should be both reusable and washable.

### Front Portion of the Outer Cover

According to examples, such as the outer cover of FIGS. 1a-1b, the back portion Sb of the reusable outer cover comprises a first and a second side portion 110, 111, and a flap member 140 as described in the above, which may be referred to as a back flap member. As described in the above, the outward facing side of the back flap member 140 comprises one or more back insert fastener areas 141, 142 for detachably fastening an insert to the outer cover.

Further, as in the illustrated outer cover, the front portion Sf of the outer cover may comprise another flap member 120, which may be referred to as a front flap member 120. The front flap member 120 may be attached to the main member 150m so as to form a pocket in the front portion thereof.

Further, the front portion Sf of the reusable outer cover may comprise one or more front insert fastener areas 121, 122 for detachably fastening an insert to the outer cover.

According to examples, as in the outer cover of FIGS. 1a-1b, the outward facing side of the front flap member 120 comprises one or more front insert fastener areas 121, 122 for detachably fastening an insert to the outer cover.

The back insert fastener areas 141, 142 and the front insert fastener areas 121, 122 may be the only insert fastener areas of the outer cover. For example, the crotch portion Sc of the outer cover may be free from insert fastener areas 121, 122, 141, 142.

According to examples, and as in the outer cover of FIGS. 1a-1b, the front portion of the outer cover comprises a set of one or more insert fastener areas 121,122, and the back portion of the outer cover comprises a stet of one or more insert fastener areas 141, 142, each insert fastener area comprising a surface fastening material for providing detachable attachment to the insert.

Each set of insert fastener areas spans a transversal fastening width oWf. The transversal fastening width may preferably be no less than a minimum transversal width Wmin of the outer cover.

### Transversal Fastening Width of Insert Fastener Areas

As mentioned in the above, the outer cover may comprise a set of back insert fastener areas 141, 142 and optionally also a set of front insert fastener areas 121, 142, which sets each spans a transversal fastening width (For simplicity, in FIGS. 1a-1b, oWf is indicated only for the back set of insert fastener areas 141, 142). The transversal fastening width may preferably be no less than a minimum transversal width Wmin of the outer cover, i.e. no less than the crotch width of the outer cover.

As such, the front set of one or more insert fastener areas and the back set of one or more insert fastener areas allow attachment of the insert in a position such that the insert is transversely held out to a sufficient width in the front and back portion, respectively, while in at least part of the crotch portion, the insert may adapt to a cup shape generally within the minimum transversal width of the outer cover.

For example, the respective transversal fastening width oWf may be no less than 110% of the minimum transversal width Wmin, such as no less than 120% of the minimum transversal width (crotch width) Wmin of the outer cover.

As such, the transversal fastening width oWf will be no less than a minimum elastic leg element spacing width as measured between the first and second elastic elements 161, 162 of the outer cover 100.

The transversal fastening width oWf may preferably be less than a maximum elastic leg element spacing width as measured between the first and second elastic elements 161, 162 of the outer cover.

As such, it may be ensured that, when an insert 200 is attached to the outer cover 100, the elastic elements 161, 162 of the outer cover assists in providing a correct position for the insert visavi the wearer.

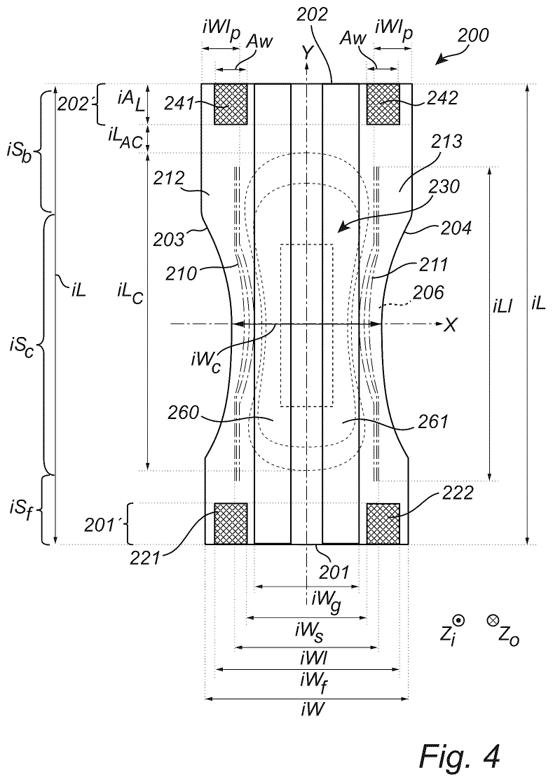

Moreover, it may be ensured that the outer cover, when in use, provides the required tension in transversal direction to the insert 200 attached thereto, so as to stretch any insert leg elastic members 210, 211 and any inner side barriers 260, 261 to their correct position. (See FIG. 4).

For example, and as in the illustrated variant, each of the front set and the back set of insert fastener areas may comprise a first subset of insert fastener areas 121; 141 and a second subset of insert fastener areas 122; 142, the first and seconds subsets being transversally spaced by a transversal spacing width oWs. To further ensure correct positioning and shaping of the insert visavi the outer cover, the transversal fastener spacing width oWs may be no less than the minimum transversal width oWc of the outer cover.

For example, the transversal spacing width oWs in the front set and in the back set may be at least 120% of the minimum transversal width.

As such, the first and second subsets of one or more insert fastener areas in the front set and the back set may ensure that the insert, when attached to the fastener areas, is extending at least over the crotch width of the outer cover.

For example, in the front set and/or in the back set, the transversal spacing width may be at least two times the transversal width of each insert fastener area, such as at least three times the transversal width of each insert fastener area, or at least four times the transversal width of each insert fastener area.

For example, in the front set and/or in the back set the transversal spacing width may be no less than 40% of the transversal fastening width, such as no less than 50% or no less than 60% of the transversal fastening width.

As such, the outer cover may be free from fastener areas over the transversal spacing width oWs. This implies that the insert may be free from attachment to the outer cover in a central transversal region, which may be beneficial for forming the insert during use. For example, the spaced arrangement may allow the insert to assume a slight cup-shape when seen in the transverse direction, improving the inserts retention capacity. Moreover, when the outer cover is free from insert fastener areas over the transversal spacing width, the surface fastening material of the insert fastener areas is provided only where needed, implying that the amount of material on the outer cover may be reduced.

Also, since the insert fastener areas may exhibit a relatively low stretchability, for example if provided by insert fastener members having a relatively low stretchability, it may be desired to limit the extension of the insert fastener areas so as to reduce their impact on the stretchability of the outer cover.

For example, in at least one, preferably each, of the front portion and the back portion of the outer cover, the set of one or more insert fastener areas spans over a transversal fastening width oWf, which is no less than the minimum transversal width Wmin, such as the transversal fastening width oWf is no less than 110% of the minimum transversal width Wmin, or no less than 120% of the minimum transversal width Wmin.

### Waist Attachment Elements and Corresponding Attachment Elements

As stated in the above, each of the first and second side portions 110, 111 comprises one or more waist attachment elements 115, 116, for detachable fastening to the other side portion 115, 116, or to a front side portion, or to the front portion of the outer cover.

According to examples, such as the outer cover of FIGS. 1a-1b, each of the first and second side portions 110, 111 comprises one or more waist attachment elements 115, 116 for detachably fastening to the front portion of the outer cover.

According to examples, such as the outer cover of FIGS. 1a-1b, the one or more waist attachment elements 115, 116 of the first side portion 110 and the second side portion 111 are arranged so as to extend no further than the waist region length WL towards the transversal axis X.

According to examples, such as the outer cover of FIGS. 1a-1b, the one or more waist attachment elements 115, 116 of the first side portion 110 and the second side portion 111 are confined to a longitudinal region of the outer cover extending along the waist region length WL.

For example, the one or more waist attachment elements 115, 116 may be arranged at the inward facing side of each of the first and second side portions 110, 111.

According to examples, such as the outer cover of FIGS. 1a-1b, the other one out of the front portion and the back portion of the outer cover, preferably (as in the illustrated example) the front portion of the outer cover, comprises one or more corresponding attachment elements 117, 118 (See FIG. 2) for detachable fastening to the waist attachment elements 115, 116. The one or more corresponding attachment elements 117, 118 may be arranged on the outward facing side of the outer cover.

According to examples, such as the outer cover of FIGS. 1a-1b, the one or more corresponding attachment elements 117, 118 may provide for detachable fastening of the waist attachment elements 115, 116 at a plurality of different positions, enabling the outer cover to assume a plurality of different waist widths as measured along the waist of the outer cover when the outer cover is in a closed position. Accordingly, the size of the waist may be adapted to the wearer, thus ensuring that the side portions 110, 111 are subject to transversal forces when the article is worn.

The waist attachment elements 115, 116 and the corresponding attachment elements 117, 118 may comprise any type of fastener area similar to those described herein in relation to the insert fastener areas of the outer cover, or to the cover fastener areas of the insert. For example, the waist attachment elements 115, 116 and the corresponding attachment elements 117, 118 may comprise mechanical and/or frictional detachable attachments by complementary surface fastening materials such as corresponding hooks and loops, or hooks and hooks.

### Back Edge and Front Edge

According to examples, such as the outer cover of FIGS. 1a-1b, the back edge 102 extends essentially parallel to the transversal axis X at least over a width spanning the minimum crotch width Wmin of the outer cover. Preferably, the back edge 102 extends essentially parallel to the transversal axis X over a width spanning the side portions of the outer cover.

According to examples, such as the outer cover of FIGS. 1a-1b, the back edge 102 extends essentially parallel to the transversal axis X, at least over the minimum crotch width Wmin) of the outer cover 100. According to examples, such as the outer cover of FIGS. 1a-1b, the back edge 102 extends essentially parallel to the transversal axis X throughout its entire width.

According to examples, such as the outer cover of FIGS. 1a-1b, the front edge 101 extends essentially parallel to the transversal axis X, at least over a width spanning the minimum crotch width Wmin of the outer cover 100. According to examples, such as the outer cover of FIGS. 1a-1b, the front edge 101 extends essentially parallel to the transversal axis X throughout its entire width.

When the front edge 101 and the back edge 102 both extend essentially parallel to the transversal axis X along their widths, a straight continuous waist edge may be formed when the outer cover 100 is worn. This may be desired for fit and comfort.

### Symmetry

According to examples, such as the outer cover of FIGS. 1a-1b, the chassis portion 150 of the outer cover may be symmetrical with reference to the central longitudinal axis Y.

According to examples, such as the outer cover of FIGS. 1a-1b, the entire outer cover may be symmetrical with reference to the central longitudinal axis Y.

### Construction of the Outer Cover

FIGS. 3a-3e show an example of parts which may together make up the outer cover of FIGS. 1a-1b.

FIG. 3a illustrates a main member 150m of the chassis portion 150 of the outer cover. The main member 150m comprises a chassis front edge 101c forming part of the front edge 101 of the outer cover 100. According to examples, and as in the Figures, the chassis front edge 101c makes up the entire front edge 101 of the outer cover.

The main member 150m further comprises a chassis portion rear edge 102c which makes up a part of the back edge 102 of the outer cover. According to examples, and as in the Figures, the back edge 102 of the outer cover will also comprise the back edges of the first side portion 110 and the second side portion 111.

The main member 150m further comprises a first chassis side edge 103c and a second chassis side edge 104c, the first and second chassis side edges 103c, 104c connecting the chassis front edge 101c and the chassis back edge 102c.

As illustrated in FIG. 3a, each of the first chassis side edge 103c and the second chassis side edge 104c may be divided into different segments 191, 193, 151; 192, 194, 152.

The first chassis side edge 103c comprises a first side attachment segment 191. The first side portion 110 is attached to the chassis portion 150 along the first side attachment segment 191. Moreover, the first chassis side edge 103 c comprises a first flap attachment segment 193. The flap member 140 is attached to the main member 150m along the first flap attachment segment 193.

Thus, and as illustrated in FIG. 3a, the first flap attachment segment 193 is a part of the first side attachment segment 191. The first side attachment segment 191 extends beyond the first flap attachment segment 193 towards the transversal axis X by a longitudinal distance D, as described in the above.

Similarly, the second chassis side edge 104c comprises a second side attachment segment 192. The second side portion 111 is attached to the chassis portion 150 along the second side attachment segment 192. Moreover, the second chassis side edge 103 c comprises a second flap attachment segment 193. The flap member 140 is attached to the main member 150m along the second flap attachment segment 193.

Thus, and as illustrated in FIG. 3a, the second flap attachment segment 194 is a part of the second side attachment segment 192. The second side attachment segment 192 extends beyond the second flap attachment segment 194 towards the transversal axis X by a longitudinal distance D, as described in the above.

The first and second side attachment segments 191, 192 extends along the side attachment length SL. The first and second flap attachment segments 193, 194 extends along a flap attachment length also being a waist region length WL.

According to examples, and as in FIG. 3a, the first and second side attachment segments 191. 192 each extends substantially parallel to the longitudinal axis Y.

Further, as seen in FIG. 3a, the first chassis side edge 103c comprises a first gathered edge segment 151, and the second chassis side edge 104c comprises a second gathered edge segment 152. According to examples, and as in the Figures, the first and second gathered edge segments 151, 152 follow substantially directly from the first and second side attachment segments 191, 192.

Elastic edge elements (see FIGS. 1a-1b) may be applied to the first chassis side edge 103c and the second chassis side edge 104c so as to gather the first and second gathered edge segments 151, 152 as described in the above.

According to examples, and as in FIG. 3a, the first and second chassis side edges 103c, 104c together define an hourglass shape of the main member 150m, such that that the main member 150m has a narrowest width along the transversal axis X in the crotch portion Sc of the outer cover 100. For example, and as illustrated, the gathered edge segments 151, 152 of the first and second chassis side edges 103c, 104c may be curved to form a narrowest width along the transversal axis X in the crotch portion Sc of the outer cover 100.

According to examples, and as in FIG. 1a and FIG. 3a, in the front/back portion Sc of the outer cover comprising the side portions 110, 111, the main member 150m has a widest width along the transversal axis X corresponding approximately to the width between the first and second side edge segments 191, 192.

FIGS. 3d and 3e illustrate the second side portion 111, comprising a first side edge, a second side edge, a back edge and a front edge. A waist attachment member 116 is arranged adjacent the second side edge. The first side edge have a side edge length being substantially equal to the side attachment length SL. Thus, the second side portion 111 is attached to the chassis portion 150 by the first side edge of the second side portion 111 being attached to the second side attachment segment 192 of the main member 150m.

In a similar manner, a first side portion 110 (not illustrated) is attached to the chassis portion 150 by a second side edge of the first side portion 110 being attached to the first side attachment segment 191 of the main member 150m.

FIG. 3b illustrates a flap member 140, in this example being a back flap member 140. The back flap member 140 comprises, as described in the above, a back edge, a front edge towards the transversal axis X, and two transversally spaced side edges. A first side edge is attached to the first flap attachment segment 193 of the main member 150m, and a second side edge is attached to the second flap attachment segment 194 of the main member 150m.

As described in the above, and as in the outer cover of FIG. 1a-b, a first joint 181 may attach the first side edge of the flap member 140 to the first flap attachment segment 193 of the main member 150m, and the side edge of the first side portion 110 to the first side attachment segment 191 of the main member 150m. Similarly, a second joint 182 may attach the second side edge of the flap member 140 to the second flap attachment segment 194 of the main member 150m, and the side edge of the second side portion 111 to the first side attachment segment 192 of the main member 150m.

In addition, one or more joints may attach the back edge of the flap member 140 to the main member 150 m, so as to form a closed pocket being open in a direction towards the transversal axis X.

According to examples, and as in the illustrated outer cover, the free edge of the flap member 140 may have a transversal width being slightly less than the transversal width between the first and second flap attachment segments 193, 194. For example, the front or back edge of the flap member may have a transversal width being greater than the edge towards the transversal axis X, i.e. the free edge. This ensures that at least the free edge of the flap member 140 is stretched such that it does not risk folding “up” when the outer cover is in use. As exemplified in FIG. 3b, the width of the flap member 140 may be tapering from a maximum width towards the front/back edge of the outer cover, to a minimum width towards the transversal axis X, i.e. at the free edge of the flap member.

As seen in FIG. 3c, the flap member 140 may further comprise a set of insert fastener areas 141, 142 arranged on the outward facing side of the flap member 140, i.e. such that the fastener material of the insert fastener areas 141, 142 faces the inward facing side of the main member 150m, as described in the above.

FIG. 3c illustrates another flap member 120, in this example being a front flap member 120. The front flap member 120 comprises a front edge, a back edge towards the transversal axis X, and two transversally spaced side edges. A first side edge may be attached to the main member 150m along a front portion of the first chassis side edge 103c by one or more joints, and a second side edge may be attached to the main member 150m along a front portion of the second chassis side edge 104c by one or more joints. In addition, one or more joints may attach the front edge of the flap member 120 to the main member 150m, so as to form a closed pocket being open in a direction towards the transversal axis X.

Similar to the back flap member 140, the free edge of the front flap member 120 may have a transversal width being slightly less than the transversal width between the front portions of the first chassis side edge 103c and the second chassis side edge 104c where it is attached. (See FIG. 1a.)

### Cloth Percentual Stretch in Construction of Outer Cover

According to examples, and as illustrated in FIG. 1b, the chassis portion 150 comprises a waist region WR extending along the waist region length WL, i.e. along the length of the flap member 140. Accordingly, in the waist region WR, the chassis portion 150 may comprise double layers of fabric (one layer from the main member 150m and one layer from the flap member 140.) As such, the waist region WR displays a Cloth Percentual Stretch which corresponds to the Cloth Percentual Stretch of the double fabric layers. (See FIGS. 1a-1b)

In contrast, an intermediate region IR extending transversally between the first and second side attachment segments 191, 192 and longitudinally along the first distance D displays a Cloth Percentual Stretch which corresponds to the Cloth Percentual Stretch of a single fabric layer.

The main member 150m may, as in the illustrated example, comprise a single layer of a fabric material. As such, between the side joints 181, 182 and extending along the distance D, there may be defined an intermediate region IR displaying a Cloth Percentual Stretch corresponding to the Cloth Percentual Stretch of the single fabric layer. The main member 150m may however, as in the illustrated example, comprise more than one fabric panel. For example, the main member 150 may comprise two fabric panels being joined by a transversal joint 183. The transversal joint 183 may extend essentially parallel to the transversal axis X, and in the crotch portion Sc of the outer cover. For example, as in the illustrated variant, the transversal joint 183 may extend between the gathered edge segments 151, 152. By means of the transversal joint 183, the stretchability of the outer cover in the crotch portion Sc may be adjusted. Accordingly, the main member 150m may, as in the illustrated example, comprise a first half and a second half, joined by the transversal joint 183. Each half may comprise one single fabric layer. Thus, as in the outer cover of FIGS. 1a-1b, the outer cover may further comprise a first crotch region CR1 extending longitudinally from the intermediate region IR to the transversal joint 183, and a second crotch region CR2 extending longitudinally from the transversal joint 183 towards the front portion of the outer cover.

The front portion of the outer cover may, as in the illustrated variant, comprise a second waist region WR2 where the main member 150m is provided with a front flap member 120.

The intermediate region IR has a first transversal Cloth Percentual Stretch, and the waist region WR has a second transversal Cloth Percentual Stretch, where the second transversal Cloth Percentual Stretch is less than the first Cloth Percentual Stretch.

In this example, the difference between a first transversal Cloth Percentual Stretch and the second transversal Cloth Percentual Stretch is achieved by the waist region WR comprising a first number of layers of a fabric material, and the intermediate region IR comprising a second number of layers of the same fabric material.

Although in other variants, the waist region WR and the intermediate region IR could comprise different fabric materials, it is advantageous in manufacturing as well as for the durability of the outer cover in washing that the waist region WR and the intermediate region IR comprises the same fabric material. In other words, the fabric material of the one or more layers of the waist region and the fabric material of the one or more layers of the intermediate region is the same fabric material.

Advantageously, and as in the Outer Cover of the drawings, all regions making up the outer cover may comprise the same fabric material.

With “same fabric material” is meant that the fabric material is the same in all material properties, such as if the layers of the same fabric materials come from the same roll of fabric material. However, it is to be understood that the color could differ between regions still being regarded to be of the “same fabric material”, provided that this color difference does not impart any other significant difference between the fabric materials being of the “same material”.

As set out in the above, the second transversal Cloth Percentual Stretch may be from 30 to 70% of the first transversal Cloth Percentual Stretch, such as from 40 to 60% of the first transversal Cloth Percentual Stretch. In the exemplified outer cover, the second transversal Cloth Percentual Stretch is from 45 to 55% of the first transversal Cloth Percentual Stretch, namely approximately 50% of the first transversal Cloth Percentual Stretch.

According to examples, and as explained in the above in connection to FIG. 3d, the first and/or second side portion 110, 111 may each comprise double layers of textile material. For example, and as illustrated in FIG. 3d, the first and/or second side portion 110, 111 may be formed by a panel of textile material being folded along a folding axis F so as to form the first/second side portion comprising two layers of fabric.

As such, the first/second side portion 110, 111 may form a first/second side region SR1, SR2 each displaying a Cloth Percentual Stretch which corresponds to the Cloth Percentual Stretch of the double fabric layers.

The double fabric layers of the first/second side region SR1, SR2 may advantageously be from the same textile fabric as the layer in the main member 150m and the flap member 140, or in the intermediate region IR and the waist region WR.

Thus, each of the first and second side regions SR1, SR2 has a side portion transversal Cloth Percentual Stretch, the side portion transversal Cloth Percentual Stretch being less than the first transversal Cloth Percentual Stretch.

Since, similar to the waist region WR, the first and second side regions SR1, SR2 comprises double layers of the fabric materials, the side portion transversal Cloth Percentual Stretch is approximately the same as the second transversal Cloth Percentual stretch of the waist region WR.

Thus, the side portion transversal Cloth Percentual Stretch is from 30 to 70% of the first transversal Cloth Percentual Stretch of the intermediate region IR, such as from 40 to 60% of the first transversal Cloth Percentual Stretch. In the illustrated example, the side portion transversal Cloth Percentual Stretch is from 45 to 55% of the first transversal Cloth Percentual Stretch, namely approximately 50% of the first transversal Cloth Percentual Stretch.

### Absorbent Insert

In use, an absorbent insert is to be detachably attached to the outer cover for forming an absorbent article. The outer cover as described herein may be adapted to be used with different absorbent insert providing for example different absorption capacities so as to adapt to the wearer's needs, and/or optionally intended for different usage such for use under daytime or night time.

The absorbent insert may in some variants be reusable, such that the insert may be cleaned or otherwise restored after having been used to absorb bodily liquids.

The absorbent insert may in some variants be disposable, such that the insert is thrown away after having been used to absorb bodily liquids. When the absorbent insert is disposable, it may in some variants be e.g. composable or partly or fully recyclable.

FIG. 4 shows an embodiment of an absorbent insert being detachably attachable to the outer cover of FIGS. 1a-1b.

FIG. 4 illustrates a variant of an insert 200, the insert being in a flat laid-out state and as seen from an inside direction of the insert It will occur to the skilled person that, since the outer cover 100 is reusable, it may be teamed with various different inserts, that may have features that look or are different from the ones depicted in FIG. 4. Some non-limiting, possible variations are already indicated in this description.

According to examples, and as in the insert of FIG. 4, the insert 200 has a liquid-permeable topsheet 205, a liquid-impermeable backsheet 206, an absorbent assembly 230. The insert 200 may, as the depicted variant, comprise inner side leakage barriers 220, 221. The insert 200 may, as the depicted variant, comprise insert leg elastic members 210, 211. The insert 200 comprises a front set of cover fastener areas 221, 222 arranged in the front portion iSf of the insert, and a back set of cover fastener areas 241, 242 arranged in the back portion iSb of the insert.

### General Construction of Absorbent Insert

### Topsheet

As stated in the above, the insert comprises a liquid impermeable backsheet, a liquid permeable topsheet, and an absorbent assembly provided between the topsheet and the back sheet.

Materials suitable for topsheets are commonly known in the art of disposable absorbent hygiene products, and for the purposes of the present disclosure any material commonly known for use as a topsheet material may be used, including, but not limited to non-woven materials and perforated polymeric films.

The topsheet is suitably sufficiently liquid permeable to allow discharged body fluids such as urine to penetrate through the thickness of the topsheet. Also, the topsheet is suitably manufactured from a material which is compliant and soft feeling to the skin of the wearer.

The topsheet may be manufactured from various web materials such as woven and nonwoven webs, perforated films, open cell foams, or combinations or laminates of the above-mentioned materials.

A nonwoven material suitable as a topsheet can be manufactured from synthetic fibres such as polyester or polypropylene, or natural fibres such as cotton fibres. A mix of synthetic and natural fibres may also be used.

The nonwoven materials to be used for the topsheet may for example be made of a spunbond, a spunbond/spunbond composite or a spunbond/meltblown composite, such as a SMS, SSMS, SSMMS, SMMS, nonwoven material of polypropylene or bicomponent fibers of polypropylene and polyethylene, or of a combination of such materials. The topsheet may also have elastic properties.

The topsheet may be hydrophilized in order to improve the tendency for urine to penetrate the topsheet into the underlying structures. Methods for hydrophilizing nonwovens are known and include coating the nonwoven material with a hydrophilic coating, such as by applying a surfactant coating; by applying a hydrophilic monomer composition and a radical polymerization initiator onto the nonwoven followed by initiating a polymerization reaction on the nonwoven; by applying a coating of hydrophilic nanoparticles; or by treating the nonwoven surface with a high energy treatment.

A surfactant coating may be obtained for example by applying a surfactant composition to the non-woven material by any suitable means including spraying, slot coating, kiss roll coating and/or soaking the material in a bath containing the surfactant. The hydrophilization treatment may be performed in-line during assembly of the absorbent hygiene product, or may performed separately and the topsheet may then delivered as ready-to-use rolls to the absorbent hygiene product manufacturing plant.

The topsheet material may have a basis weight of from 8 to 20 g/m², such as from 12 to 17 g/m². However, the disclosure is not limited to topsheet materials having this basis weight only.

### Backsheet

Materials suitable as backsheets are commonly known in the art of absorbent hygiene products. The backsheet may be substantially impermeable to liquids, such as urine.

The backsheet may be breathable. A breathable back sheet is beneficial for the comfort and skin health of the wearer. As the insert disclosed herein is intended for use with an outer cover as disclosed herein to form an absorbent assembly, use of a breathable backsheet may enable an absorbent assembly to be formed, which assembly exhibits satisfactory breathability.

The backsheet may be substantially liquid impermeable and breathable, implying that air and other gases may pass through the backsheet while being substantially impermeable to liquids.

For the purposes of the present disclosure, any material commonly known for use as a backsheet material may be included in the backsheet, including but not limited to polymeric films, for example films of polyethylene, polypropylene or copolymers of polyethylene or polypropylene, hydrophobized nonwoven materials, fluid impermeable foams and fluid impermeable laminates.

The backsheet may comprise one or more layers of material. For example, the backsheet may be a laminate of a liquid impermeably polymeric film towards the absorbent assembly and nonwoven towards the garment side, to provide a textile, soft feeling to the outward facing surface of the absorbent hygiene product.

It is also contemplated that the backsheet may be made or otherwise include an entirely or partially elastic material in order to give the product a better fit when in use.

However, with the present insert, it is believed to be advantageous if the backsheet comprises a nonwoven, for example that the backsheet is a laminate of a porous polymeric film and a nonwoven.

### Absorbent Core

The absorbent assembly can comprise one or more absorbent cores. The cores can be constructed from one or more layers of cellulose fluff pulp. The cellulose fluff pulp can be mixed with fibres or particles of a highly absorbent polymer material, so-called superabsorbent polymers, of the type that chemically binds large quantities of fluid on absorption with the formation of a fluid-holding gel. The absorbent core can also comprise highly absorbent polymer material arranged in a layer inside the absorbent core or connected to the surface or surfaces of the absorbent core. The absorbent core can further include further components for improving the properties of the absorbent core. Examples of such components are binding fibres, various types of fluid-dispersing layers or fibres, dimensionally stabilising components, reinforcing fibres or the like.

Superabsorbent polymers are well-known in the field of absorbent products and are used to help improve the absorbent properties of such products. Superabsorbent polymers are constituted by water-swellable and water-insoluble polymers that are capable of absorbing large quantities of fluid upon formation of a hydrogel, such as capable of absorbing at least 5 times their weight of an aqueous 0.9% saline solution as measured according to the method NSWP 241.0.R2. The superabsorbent polymer polymers for use in accordance with the present disclosure may be inorganic or organic crosslinked hydrophilic polymers, such as polyvinyl alcohols, polyethylene oxides, crosslinked starches, guar gum, xanthan gum, crosslinked polyacrylates, and the like. The polymers may be in the form of powders, granules, microparticles, films, foams and fibers, for example. Upon contact with fluids, such super absorbent polymers swell by absorbing the fluids into their structures. In general, super absorbent polymers can quickly absorb fluids insulted into such products, and can retain such fluids to prevent leakage and help provide a dry feel even after fluid insult.

The type of super absorbent polymer used in an absorbent assembly of the embodiments discussed herein may be the same or may vary within the assembly. For example, a super absorbent polymer with a first set of characteristics may be used in the front and back regions of the absorbent assembly, or in a first absorbent core, and a super absorbent polymer with a second set of characteristics may be used in the central region of the absorbent assembly, or in a second absorbent core. The characteristics referred to in this section is for example the centrifuge retention capacity, absorption under load and/or the gel layer permeability.

Thus, the absorbent assembly of the insert as proposed herein may advantageously comprise super absorbent polymers. For example, the absorbent assembly may comprise super absorbent polymers and cellulosic fibers.

For example, the absorbent assembly may comprise a super absorbent polymer concentration of at least 15 weight %, based on the total weight of cellulosic fibers and super absorbent polymer.

The absorbent assembly may comprise a first absorbent layer and a second absorbent layer, the first and second absorbent layers being stacked in between the backsheet and the topsheet.

As such, super absorbent polymers may be present in one or both of the first and second absorbent layers.

### Acquisition Layer

Further, the insert may include an acquisition layer applied between the topsheet and the absorbent assembly. Materials suitable as acquisition layers, also referred to in the art as transfer layer, or ADL, are commonly known in the art of absorbent hygiene products, and for the purposes of the present disclosure, any material known to the person skilled in the art as being useful as an acquisition layer may be used. An acquisition layer may for example be in the form of an airlaid layer, a spunlace layer, a high-loft, foam or any other type of material layer which may be used in an absorbent hygiene product to act as a liquid acquisition and absorption layer. The acquisition layer is suitably adapted to quickly receive and temporarily store discharged liquid before it is absorbed by the absorbent core. Such acquisition layer may be composed of for example airlaid nonwoven, spunlace nonwoven, high loft nonwoven or foam materials. An airlaid nonwoven may be produced with fluff, wood pulp, and here the fluff fibres are dispersed into a fast-moving air stream and condensed onto a moving screen by means of pressure and vacuum. The acquisition layer may preferably be of an air-through bonded nonwoven of polyester fibers.

### Further Properties of the Insert Relating to the Absorbent Assembly

The absorbent assembly may be mainly positioned in the crotch portion of the insert and have an absorbent assembly length that spans no more than 70% of the longitudinal length of the insert, such as no more than 85% of the longitudinal length of the insert.

Thus, the absorbent assembly does not extend all the way to the front and back edge of the insert, preventing liquid from being stored close to the edges which are positioned inside the flap members of the outer cover. This reduces the risk of soiling of the outer cover, allowing the outer cover being reused without the need of being cleaned.

For example, the cover fastener areas of the front set and of the back set may be confined to the portions of the insert extending longitudinally beyond the absorbent core towards the front edge or back edge, respectively. As such, the attachment of the insert to the outer cover is constant, regardless of whether the insert has absorbed any amount of liquid or not.

The absorbent core may be hour-glass shaped, having a narrowest width in the transverse direction in a longitudinal central position of the crotch portion of the insert.

With hour-glass shaped is meant herein that the absorbent core has a narrowest width in the crotch portion of the insert, and that a width of the absorbent core towards the front portion and towards the back portion, respectively, is greater than the narrowest width.

In other alternatives, the absorbent core may have other shapes, such as a rectangular or oval shape.

Optionally, the longitudinal edges of the insert are partially corresponding to the hour-glass shape of the absorbent assembly.

As such, the insert may have a shape with a narrowest width in the transverse direction in the crotch portion of the insert.

In other alternatives, the insert may have a rectangular shape.

The insert may have a length along the longitudinal axis being greater than a width along the transversal axis.

For example, the insert may have total absorption capacity of at least 1000 ml, such as from 1000 to 4000 ml. As such, the insert is an insert suitable for use as in an absorbent article being an incontinence article suitable for adult wearers.

In some variants, the absorbent assembly is arranged to be symmetric in view of a central transversal axis of the insert. As such, regardless of the front/back orientation of the insert visavi a wearer, the insert will provide similar absorbent functions.

In some variants, the absorbent assembly is arranged to be non-symmetric in view of a central transversal axis of the insert. As such, depending on the front/back orientation of the insert visavi a wearer, the insert may display different absorbent functions. For example, the absorbent assembly may provide for more absorption capacity in a region between the central transversal axis and the back edge of the insert than in a region between the central transversal axis and the front edge of the insert.

The insert 200 extends along a longitudinal direction Y and a transverse direction X. The topsheet 205 forms part of the innermost surface of the insert 200 facing in an inward direction Zi and which is intended to be directed towards the wearer when the absorbent article is worn. The backsheet 206 is forming at least part of the outermost surface of the insert 200, facing in an outward direction Zo. The absorbent assembly 230 is enclosed between the topsheet 205 and the backsheet 206. The backsheet may cover the entire surface area of the insert 200, that is, the backsheet forms the outermost surface of the insert 200. Thus, the width iW and the length iL of the insert 200 maybe equal to the width and length of the backsheet.

The liquid-permeable topsheet 205 can consist of any material that is suitable for the purpose. Examples of commonly encountered liquid-permeable topsheet materials are nonwoven materials, perforated plastic films, meshes made of plastic or textile, and liquid-permeable foam layers. Liquid-permeable covering materials which consist of continuous thin fibers that extend predominantly in the longitudinal or transverse direction of the article are also encountered. Laminates consisting of two or more of the above-mentioned possible covering materials are also commonly encountered, as are coverings consisting of different materials in different parts of the surface.

The liquid-impermeable backsheet 206 may extend beyond the absorbent assembly 230 around the periphery of the entire absorbent assembly 230. The backsheet can consist of a range of different materials. The backsheet may consist of a thin and liquid-impermeable plastic film, although it is also possible to use other types of liquid-impermeable material such as nonwoven materials that have been made liquid-impermeable, for example by means of plastic coating, liquid-tight foam layers, liquid-tight adhesive or similar. The backsheet can also consist of a liquid-impermeable, vapor-permeable material, i.e. a liquid impermeable and breathable material.

The innermost layer of the insert 100 comprising the liquid-permeable topsheet 205 is attached to the backsheet 206 outside the absorbent assembly 230 along the entire periphery of the absorbent assembly 230. The innermost layer comprising the liquid-permeable topsheet 205 and the backsheet may be attached to one another by a number of different means. Examples of means of attachment include gluing, thermal fusion, ultrasonic welding or the like.

The absorbent assembly 230 may, as depicted, have a length and width that are smaller than the innermost layer comprising the topsheet 205 and the backsheet 206, thus being present in a central region of the insert 200 where liquid and feces exit the body of the wearer. This results in a less bulky, more comfortable design, thereby being preferred. However, it will occur to the skilled person that alternative sizes, shapes and configurations of the absorbent assembly 230 may also be suitable for use in an absorbent article as presented herein. As illustrated in FIG. 2a, the absorbent assembly 230 may have a longitudinal length iLc.

For example, and as illustrated in the drawings, the absorbent assembly 230 may have an hourglass-shape, which is intended to be situated at the narrowest area between the wearer's thighs when being worn. The longitudinal side edges 203, 204 of the innermost layer comprising the topsheet 205 and of the backsheet 206 also correspond to this hour-glass shape. This results in the insert being more formfitting and thus more comfortable to wear than if it were to have a rectangular shape.

The absorbent assembly 230 may, as in the illustrated variant (FIG. 4) comprise a first layer 231 and a second layer 232. Further, the absorbent assembly may comprise a receiving layer 233. The receiving layer 233 is positioned directly below the topsheet 205 and is adapted to rapidly receive quite large quantities of liquid and to retain this liquid temporarily, in order subsequently to release the temporarily stored liquid to other parts of the absorbent assembly 230. Alternative configurations of the absorbent assembly 230 may be envisaged and both the second layer 232 and the receiving layer 233 may be considered optional, independent from one another. The absorbent assembly as such may be constructed similarly to known absorbent assemblies and as such can be constructed from one or more layers of cellulose fluff pulp. The cellulose fluff pulp can be mixed for this purpose with fibers or particles of a high-absorbency polymer material, known as superabsorbents, of the kind which, in conjunction with absorption, chemically bonds large quantities of liquid to form a liquid-containing gel. The absorbent assembly 230 can also contain high-absorbency polymer material arranged in a layer inside the absorption body or in conjunction with the surface or surfaces of the absorption body. Additional components to improve the characteristics of the absorbent assembly 230 can also be present in the absorbent assembly 230. Examples of such components include binding fibers, different types of liquid-distributing layers or fibers, form-stabilizing components, reinforcing fibers or the like. The absorbent assembly 230 can naturally also consist of other types of absorption material, such as absorbent nonwoven material, absorbent foam, textile materials, peat or mixtures of different kinds of absorption material.

As central transversal axis X as illustrated in FIG. 2a divides the insert 200 into a front half and a back half.

In some variants, the insert 200 may be symmetric as seen over the central transversal axis X. Thus, the absorbent assembly 230 may be symmetric as seen over the central transversal axis X of the insert 200. In this case, the insert 200 may provide for essentially the same absorbing function in the front half and in the back half of the insert 200.

In some variants, the insert 200 may be asymmetric as seen over the central transversal axis X. Thus, the absorbent assembly 230 may be asymmetric as seen over the central transversal axis X of the insert 200. In this case, the insert 200 may provide for different absorbing function in the front half and in the back half of the insert 200.

### Insert Leg Elastic Members

As depicted in FIG. 4, the insert 200 may comprise insert leg elastic members 210, 211. The insert leg elastic members 210, 211 are arranged in between the longitudinal outer edges of the absorbent assembly 230 and the longitudinal outer edges 203, 204 of the insert 200 on both sides of the insert 200, running essentially in the longitudinal direction of the insert 200. As depicted in FIG. 4, the insert leg elastic members 210, 211 may be partially curved. For example, the insert leg elastic members may be partially curved so as to follow the shape of the absorbent assembly 230, when the absorbent assembly is hour-glass shaped. A maximum transversal width over the insert leg elastic members 210, 211 is defined as the leg elastics width iWl. As illustrated in the Figures, the transversal width over the insert leg elastic members 210, 211 is the width measured in a transversal direction including both insert leg elastic members 210, 211 in the pair of insert leg elastic members 210, 211. The transversal width over the insert leg elastic members 210, 211 may, as in the illustrated embodiment, vary along the length of the insert leg elastic members 210, 211. The maximum transversal width over the leg elastic members 210, 211 in each of the front portion iSf or back portion iSb of the insert is the maximum transversal width which may be found along the length of the insert leg elastic members 210, 211 in the front or back portion iSf, iSb, respectively.

The insert leg elastic members 210, 211 are included in the insert 200 for preventing liquid and faeces from leaking out past the longitudinal edges of the insert, ensuring a close fit around the legs of a wearer. Thus, the inner leg elastic members 210, 211 contribute to the insert 200 being leakproof when used to form the absorbent article 1, which allows the entire outer cover 100 to be made of liquid-permeable material. Each of the insert leg elastic members 210, 211 may consist of one or more elastic elements, such as elastic threads, that have been applied in their stretched state between the innermost layer comprising the liquid-permeable topsheet 205 and the backsheet. The pair of insert leg elastics members 210, 211 have a longitudinal leg elastics length iLl, which may extend along at least 60% of the length of the insert. For example, the leg elastics length may be approximately coinciding with the length iLc of the absorbent assembly 230. The elastic members 210, 211 may be attached to the backsheet and the topsheet 205 by gluing, ultrasonic welding or the like.

As illustrated in FIG. 4, in each of the front portion iSf and the back portion iSb of the insert 100, the insert 100 has a maximum width iW, and the leg elastics width iWl being the maximum transversal width between the leg elastic members 210, 211 is smaller than the maximum width Wᵢ of the insert and larger than a transversal maximum width of the absorbent assembly 230.

A leg edge portion 212, 213 is defined between each insert leg elastic member 210, 211 and the adjacent side edge 203, 204 of the insert 200, in each of the front and back portions iSf, iSb of the insert 200. The leg edge portions 212, 213, may have a maximum width along the respective insert leg elastic member 210, 211 being at least 1 cm, such as at least 2 cm. The leg edge portions 212, 214 provide additional fluid leakage protection as well as increased comfort to the wearer.

### Insert Inner Side Leakage Barriers

In order further to prevent liquid or faeces from leaking out via the side edges of the insert 200, the insert 200 may be provided with a pair of inner side leakage barriers 260, 261 on the inner surface, as depicted in FIG. 4.

One inner side leakage barriers 260, 261 is arranged on either side of the central longitudinal axis Y and the inner side leakage barriers 260, 261 extend essentially in the longitudinal direction of the insert 200. The inner side leakage barriers 260, 261 comprise raisable parts which, when the insert is in use, raise in an inward direction from the topsheet 205, so as to form barriers against side leakage. The raisable parts of the inner side leakage barriers 260, 261 extend at least over a part of the crotch portion iSc of the insert 200.

A maximum transversal barrier width iWg is measured between the attached edges 260b, 261b of the raisable parts of the pair of inner side leakage barriers 260, 261. The maximum transversal barrier width iWg may be smaller than a maximum transversal width iWl over the insert leg elastic members 210, 211, if present. As in the illustrated variant, the maximum transversal barrier width iWg may be larger than a minimum transversal width of the absorbent assembly 230. In some variants, the maximum transversal barrier width iWg may optionally be larger than a maximum transversal width of the absorbent assembly 230. The respective inner side leakage barrier 260; 261 may be executed from a separate material strip, which exhibits two essentially parallel longitudinal edges. The material strip may be double-folded, in conjunction with which the parallel longitudinal edges are arranged adjacent to one another. At least a longitudinal edge of each material strip is attached to the innermost layer comprising the topsheet 205 and constitutes the attached edge 260b, 261b of the respective inner side leakage barrier 260, 261. As in the illustrated variant, a portion of the material strip may extend over the inside of the insert 200 all the way from the attached edge 260b, 261 to the adjacent longitudinal side edge 203, 204. The folded edge of the material strip constitutes the free edge of the inner side leakage barrier 260; 261. The inner side leakage barriers 260, 261 are folded down over the innermost layer, i.e. over the topsheet 205, during manufacturing and storage, and have a raised configuration during use, in which at least in the crotch portion iSc of the insert, the free edge is lifted up from the innermost layer comprising the topsheet 205.

In the depicted insert 200, the longitudinal front and back ends of the material strips are attached to the innermost layer comprising the topsheet 205 on the front edge 201 and the back edge 202 of the insert 200. Thus, the longitudinal front and back ends are not raisable.

The inner side leakage barriers 260, 261 further comprise elastic elements 260a, 261a attached to the inner side leakage barriers 260, 261 in a pre-tensioned state. The elastic elements 260a, 261a are conveniently arranged adjacent to the free edges of the inner side leakage barriers 260, 261, for example inside the folds of the material strips as in the illustrated variant. When the pre-tensioned elastic elements 260a, 261a are released, they contract together with the free edges of the inner side leakage barriers 260, 261, thereby causing the inner side leakage barriers 260, 261 to be brought into a raised configuration remote from the liquid-permeable topsheet 205. For example, the inner side leakage barriers 260, 261 may be brought into a raised position at least in crotch part of the insert corresponding to at least 60% of the longitudinal length of the insert, the crotch part being spaced from both the front end 201 and the back end 202 of the insert.

The inner side leakage barriers 260, 261 form raisable parts where the inner side leakage barriers 260, 261 may be brought to the raised configuration over a majority of the longitudinal length of the insert 200. The inner side leakage barriers 260, 261 may be attached to the topsheet 205 in attached regions adjacent the front end and the back end, respectively of the insert. In the attached regions, the material strip forming each inner side leakage barrier 260, 261 is attached to the topsheet, e.g. via a weld or an adhesive, such that, in the attached regions, the inner side leakage barriers are not raisable. For example, the inner side leakage barriers 260, 261 may be attached to the inward facing side of the insert 200 in the front edge portion 201′ and/or in the rear edge portion 202′ of the insert.

Alternatively, the longitudinal ends of the material strips may be unattached to the innermost layer, such that the free edges of the material strips forming the inner side leakage barriers 220, 221 are free to raise up from the innermost layer upon bending of the insert into the use position, eventually without the help from pre-tensioned elastic elements.

To reliably and comfortably hold the insert 200 in the outer cover 100, forming the absorbent article 1, the insert 200 and outer cover 100 are detachably attachable to one another.

### Cover Fastener Areas of Insert

Hereto, as illustrated in FIG. 4, the insert 200 comprises a back set of cover fastener areas 241, 242 in the back portion iSb and a front set of cover fastener areas 221, 222 in the front portion iSf.

Each cover fastener area 221, 222, 241, 242 comprises a surface fastening material for providing detachable attachment of the insert to the outer cover. As will be described in more detail in the below, all of the cover fastener areas 221, 222, 241, 242 in the same front or back set need not comprise the same surface fastening material. Instead, in accordance with the present disclosure, and as will be described more in the below, the cover fastener areas in the same front or back set comprise different surface fastening materials, i.e. a first surface fastening material and a second surface fastening material.

In the illustrated variant, the back set of cover fastener areas in the back portion iSb and the front set of cover fastener areas in the front portion iSf each comprises two transversally spaced cover fastener areas 241, 242; 221, 222. However, other variants are possible, such that at least one of the sets of cover fastener areas comprising more than two cover fastener areas, for example four cover fastener areas.

Regardless of the amount of cover fastener areas comprised in a set, a transversal fastening width iWf may be defined as the transversal width spanned by the set of one or more cover fastener areas.

In the illustrated variant, each one of the cover fastener areas in the front set, and each one of the cover fastener areas in the back set is arranged at the same longitudinal location. Each one of the cover fastener areas moreover have the same length iAl as seen in the longitudinal direction. However, other variants are possible, wherein for example at least one cover fastener area in a front/back set is arranged at a different longitudinal location than another cover fastener area in the front/back set. Still, the set of one or more cover fastener areas spans a longitudinal length iAl along the longitudinal axis L. However, it may be preferred that each one of the front set and the back set of cover fastener areas is symmetrical as seen over the longitudinal central axis Y.

A front edge portion 201′ is defined as the portion of the insert 200 extending from the front edge 201 of the insert 200 and over the longitudinal length iAl spanned by the front set of one or more cover fastener areas 221, 222 in the front portion iSf, and a back edge portion 202′ is defined as the portion of the insert 200 extending from the back edge 202 of the insert 200 and over the longitudinal length iAl spanned by the back set of one or more cover fastener areas 241, 242 in the back portion iSb.

Each one of the front edge portion 201′ and/or the back edge portion 202′ may extend over an edge portion length being for example less than 20% of the longitudinal length of the insert, such as less than 10% of the longitudinal length of the insert.

As such, it may be ensured a large portion of the inward facing surface of the insert 200 remains available for directly contacting the wearer's genital area and thus limiting the risk of soiling of the outer cover 100. As exemplified in the illustrated embodiment, in the longitudinal direction Y, each of the cover fastener areas 221, 222, 241, 242 in the set may be positioned at, i.e. be directly adjacent, the front edge 201 or the back edge 202, respectively. This is an advantage in manufacturing, as one piece of a fastener member material may be arranged over two subsequent inserts when formed on a continuous insert material line. In this instance, one cutting operation will separate the piece of fastener member material into two at the same time as the continuous insert material line is cut into separate inserts. In this case, the longitudinal length iAl spanned by the set of cover fastener areas will correspond to the longitudinal length of the respective edge portion 201′, 202′ of the insert 200. For example, each set of one or more cover fastener areas 221, 222; 241, 242 may span a longitudinal length iAl being less than 20% of the longitudinal length of the insert, such as less than 10% of the longitudinal length of the insert.

In the transverse direction X, each set of cover fastener areas 221, 222; 241, 242 in the front and back portion iSf, iSb, respectively, extends over a transversal fastening width iWf. As exemplified in the drawings, the transversal fastening width iWf may be no less than, and preferably greater than, the leg elastics width iWl.

This particular positioning allows for the insert to be sufficiently tensioned when the absorbent article 1 is put on a wearer, ensuring reliable functioning of the insert leg elastic members 210, 211.

The insert leg elastic members 210, 211 extend along a longitudinal insert leg elastics length iLl. The front edge portion 201′ and the back edge portion 202′ may advantageously be arranged so as not to overlap with the longitudinal insert leg elastics length iLl. In other words, each set of one or more cover fastener areas is confined to a front edge portion 201′ or a back edge portion 202′ not spanned by the leg elastics length iLl.

The transversal fastening width iWf may, as in the illustrated embodiment, extend over at least part of the leg edge portions 212, 213 of the insert, so as to ensure that the leg portions are automatically folded outward when the insert is attached to an outer cover. For example, the transversal fastening width may span into at least 50% of the transversal width of each leg portion, or at least 80% of the transversal width of each leg portion.

As in the illustrated example, in the back portion iSb, the back set of one or more cover fastener areas comprises a first subset of one or more cover fastener areas 241 being arranged on a first transversal side of the longitudinal axis Y, and a second subset of one or more cover fastener areas 242 being arranged on a second transversal side of the longitudinal axis Y. Similarly, in the front portion iSf, the front set of one or more cover fastener areas comprises a first subset of one or more cover fastener areas 221 being arranged on a first transversal side of the longitudinal axis Y, and a second subset of one or more cover fastener areas 222 being arranged on a second transversal side of the longitudinal axis Y.

In the back portion iSb, the first subset of one or more cover fastener areas 241 is spaced from the second subset of one or more cover fastener areas 242 by a transversal spacing width iWs. Similarly, in the front portion iSf, the first subset of one or more cover fastener areas 221 is spaced from the second subset of one or more cover fastener areas 222 by a transversal spacing width iWs. As such, in the front portion iSf as well as in the back portion iSb, the insert 200 is free from cover fastener areas over a spacing width iWs as measured between the subsets in the front or back portion, respectively.

This contributes to the correct shaping of the insert in use, since the insert is to be unattached to the outer cover over the transversal spacing width. Moreover, by providing cover fastener areas substantially only at the locations which are most critical to ensure the correct positioning of the insert in the outer cover, correct assembly of the insert and an outer cover may be facilitated.

The transversal spacing width iWs may for example be at least 70% of the transversal fastening width iWf. In the illustrated embodiment, each first or second subset of one or more cover fastener areas comprises one single cover fastener area. As such, the first and second subsets of one or more cover fastener areas in each of the front and back set together form a pair comprising two cover fastener areas (one cover fastener area in each subset) being spaced by the spacing width iWs.

As illustrated in the examples of the Figures, the cover fastener areas 221, 222, 241, 242 may be provided on the inward facing of the insert 200, each at a respective corner of the insert 200, for detachably attaching the insert 200 to the outer cover 100. The cover fastener areas 221, 222, 241, 242 are located outside the crotch portion iSc, thereby preventing any overlap of the attachment to the outer cover 100 with the crotch portion oSc and thus reducing the risk of soiling of the outer cover. The cover fastener areas 221, 222, 241, 242 together may cover less than 5% of the total surface area of the innermost layer comprising the topsheet 205. This is found to be sufficient for reliably attaching the insert inside the outer cover 100, thus using more material would result in unnecessary waste. Moreover, this implies that a relatively small portion of the innermost layer of the insert is occupied by the cover fastener areas 221, 222, 241, 242, and hence a relatively large portion of the innermost layer of the insert may be available for receiving bodily exudates when the absorbent article 1 is in use.

### Absorbent Article

FIG. 5 illustrates an example of an absorbent article 1, having reusable outer cover 100 and insert 200 being detachably attached to the outer cover 100. In this example, the reusable outer cover 100 is the outer cover of FIGS. 1a-3e, whereas the insert 200 is the insert of FIG. 4.

The absorbent article 1 exhibits a longitudinal direction X and transverse direction Y and has a thickness direction Z, which, when the article is worn, defines an inward direction Zi towards the wearer's skin and an outward direction Zo away from the wearer's skin. According to examples, and as in the depicted embodiment, both the outer cover 100 and the insert 200 are substantially symmetrical around a longitudinal central axis Y.

As depicted in FIGS. 1a-1b, in the absorbent article 1, the insert 200 is detachably attached to the outer cover 100, such that the insert 200 at least partially covers the inward facing side of the outer cover 100, and the outward facing side of the outer cover 100 forms an outermost surface of the absorbent article 1. Thus, an innermost surface of the outer cover 100 faces an outermost surface of the insert 200, such that for the opposite surfaces holds that, during use, an outward facing surface of the outer cover 100 is intended to face away from the wearer and the inward facing surface of the insert 200 contacts the wearer's skin. In the depicted absorbent article 1, a substantial part of both the innermost surface of the outer cover 100 and the innermost surface of the insert 200 are visible, together forming the innermost surface of the absorbent article that contacts the wearer's skin during use.

It will be understood that in the absorbent article 1, the insert 200 is detachably attached to the outer cover 100 by the by the cover fastener areas 221, 222; 241, 242 of the insert 200 being detachably attached to the insert fastener areas 121, 122; 141, 142 of the outer cover 100.

Preferably, the dimensions of the outer cover 100 and the insert 200 are such that, when the insert 200 is detachably attached to the outer cover 100, the front and back flap members 120, 140 do not overlap with the absorbent assembly 230.

For example, each flap member 120, 140 may have a longitudinal flap length being such that, in the use position, when the insert 200 is attached to the outer cover 100, the flap member 120, 140 extend(s) over less than 20% of the insert longitudinal length, preferably over less than 10% of the insert longitudinal length.

For example, the total area of the front and back flap members 120, 140 may cover less than 80%, preferably less than 85%, of a total insert area. The front and back flap members 120, 140 may be sized such that less than 15% of the total insert area is covered during use, preferably less than 10%.

Further, it is to be understood that the absorbent insert of the absorbent article may comprise any of the features as set out in the above in relation to the description of the absorbent insert 200, and the present disclosure is to encompass all such absorbent articles.

For example, the absorbent insert may comprise a backsheet being liquid-impermeable.

For example, the absorbent insert may be disposable.

### Use-Cycle of an Absorbent Article

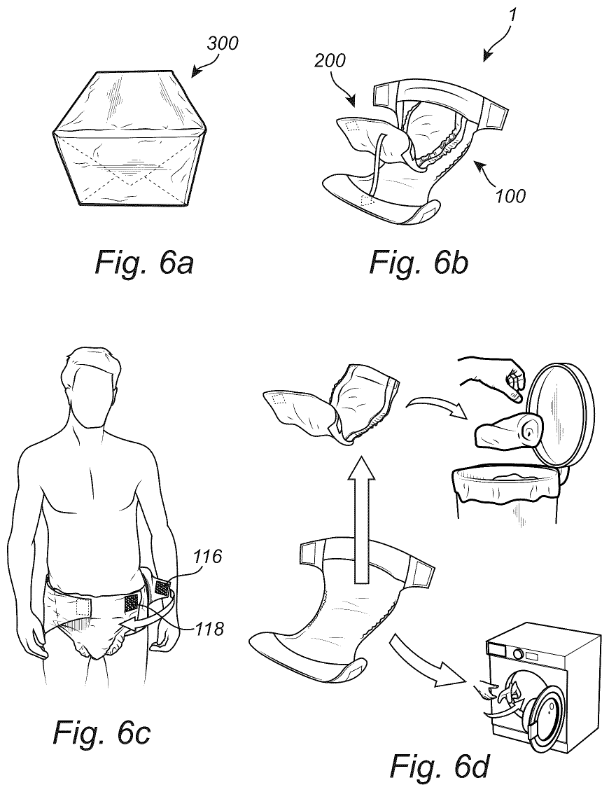

FIGS. 6a, 6b, 6c and 6d depict the use-cycle of an absorbent article 1 similar to the article depicted in FIG. 5, respectively showing a package 300 containing at least an insert 200, the attachment of an insert 200 into an outer cover 100, the absorbent article being worn by a wearer and the removal and disposal of an insert 200 and cleaning of the outer cover 1.

The package in FIG. 6a may be available containing a predetermined amount of inserts and/or one or more outer covers. In FIG. 6b is shown that first the insert 200 is detachably attached inside the corresponding flap members of the outer cover 100. Attachment is performed as described in the above. Next, the absorbent article is put on a wearer, as depicted in FIG. 6c. For the particular embodiment of the outer cover 100, side portions 110, 111 are arranged on the back end part of the article and attached to the front end part of the article, on the outward facing surface of the outer cover 100, when the article is put on a wearer. Preferably, the outer cover 100 is provided with complementary attachment means 117, 118 (See FIG. 2) at the front end part, on the surface facing away from the wearer, as depicted. The complementary attachment means 117, 118 are adapted for receiving the attachment means 115, 116 (See FIGS. 1a-1b) provided on the first and second side portions 110, 111 to ensure good detachable attachment without potentially causing damage to the main material forming the outward facing surface of the outer cover 100.

After use, the entire absorbent article 1 is removed from the wearer by detaching the first and second side portions. Thereafter, the insert may be separated from the outer cover as depicted in FIG. 6d.

For example, and as inn the present embodiment, the insert 200 is a disposable insert. Thus the insert 100 may be thrown away after use.

The outer cover 100 is preferably washable, such that it may be washed as depicted when required.

In a following use-cycle, the same outer cover 100 is then used with a new, another, insert 200.

It will be understood that the described use-cycle may be the same for many variants of the absorbent articles, independent from the particular shape of the outer cover and the insert. Furthermore, it will be understood that the use-cycle will be substantially the same for variants of absorbent articles according to the present description where the insert is reusable, apart from the insert being washed prior to being re-used in the following use-cycle, rather than discarded.

The present invention has been described with reference to a number of explanatory embodiments as shown in the figures. Obvious modifications and alternative implementations of some parts or elements will be apparent to those skilled in the art upon reading and understanding the foregoing detailed description. In particular, it will be obvious to the skilled person that various alternatives explicitly mentioned in the detailed description may be implemented independently from other explicitly mentioned alternatives that are not explicitly described as being related thereto. The invention is intended to be construed as encompassing all such modifications and alternative implementations to the extent that they fall within the scope of the appended claims.

### Test Method for Cloth Stretchability—Cloth Percentual Stretch

The following procedure is used for determining cloth stretchability.

A representative article is taken from an unopened package, and the cloth region under investigation is arranged perfectly flat (but not stretched). Samples are then cut from the directions of interest, preferably using a sharp punching tool. The samples should be representative for the studied cloth region, why obviously irregular features (such as seams or fastening elements) should be avoided. A standard sample is 30 mm wide, and more than 40 mm long (some extra material is required in the length direction for attachment into clamps, as will be further explained below).

Before testing the samples are conditioned for at least 24 hours in an environment set to 23° C. and 50% relative humidity, and subsequent testing takes place in this same environment.

A tensile tester is utilized for the measurement. Suitable machines are available e.g. from the Instron, Lloyd or Zwick companies. The tester is provided with a lower stationary clamp, vertically aligned with an upper clamp that can be raised at a constant rate (in this instance 300 mm/min). The clamps must be as wide or wider than the tested sample.

The clamps are set 40 mm apart (the sample gauge length). Slipping in the clamps must be prevented, for example by attaching tape with a high friction backing immediately adjacent to the tested 40 mm section (onto the whole sample area that is attached into the clamps). Tape can also be used to extend the sample length in order to facilitate insertion into the clamps.

The tensile tester is zeroed. The sample is then arranged into the clamps so that it rests vertically and free from wrinkles, and such that an ever so slight pretension of about 0.03 N (i.e. 0.01 N per sample width cm) can be read on the force display when starting the test.

The test is initiated, and the sample elongation is read when a force of 3 N has been reached for the standard sample. Stretchability is then calculated:

(Sample length at 3N−Initial sample length)/Initial sample length×100

The result is expressed as percentual stretch at a force of 1 N per cm of sample width-herein defined as “Cloth Percentual Stretch”

For example, if a force of 3 N is read when a standard test section (40 mm long and 30 mm wide) has been elongated with 20 mm (the clamps are now 60 mm apart), the elongation at a force of 1 N per cm of sample width is determined to 50%. Accordingly, the Cloth Percentual Stretch is 50%.

In case the cloth region of interest is small, and a sample of standard dimensions cannot be cut, samples as large as possible should be obtained. In this case, the length to width ratio (1.33) of the tested section must be maintained, and the reading force is adjusted accordingly (e.g. a 27 mm long test section should have a width of 20 mm, and percentual stretch should be read at 2 N).

1. A reusable outer cover for forming an absorbent article wherein an absorbent insert is adapted to be detachably attached to the outer cover, the outer cover having a front portion to be directed towards a front of a wearer, a back portion to be directed towards a back of a wearer, and a crotch portion extending between the front portion and the back portion, the outer cover extending along a longitudinal central axis, and along a transversal central axis, which is perpendicular to the longitudinal axis, the transversal central axis dividing the outer cover into a front half and a back half, and a thickness axis is defined perpendicular to both the longitudinal and transversal axes, wherein inward direction is defined along the thickness axis being the direction towards a wearer when the outer cover is worn, and an outward direction is defined along the thickness axis being the direction away from the wearer when the outer cover is worn,
the outer cover comprising a chassis portion extending along the longitudinal axis from a front chassis edge forming part of the front edge of the outer cover to a back chassis edge forming part of the back edge of the outer cover,
the chassis portion comprising a first chassis side edge and a second chassis side edge respectively extending between the chassis front and back edges of the chassis portion, on opposite sides with respect to the longitudinal axis, wherein,
in at least one out of the back half and the front half of the outer cover,
the outer cover comprises
a first and a second side portion extending transversally on opposite sides of the chassis portion with respect to the longitudinal axis, wherein each of the side portions of the outer cover comprises one or more waist attachment elements to attach the outer cover around the waist of a wearer during use, the first side portion is permanently attached to the chassis portion along a first side attachment segment of the first chassis side edge, and the second side portion is permanently attached to the chassis portion along a second side attachment segment of the second chassis side edge, the first and second side attachment segments defining a side portion length from the longitudinal ends of each segment closest to the central transversal axis and to the adjacent back edge or front edge of the outer cover,
and
the chassis portion comprises a main member and a flap member, the main member forming the front or back chassis edge, the first chassis side edge, and the second chassis side edge in the front or back half, and
the flap member being arranged with an outward facing side of the flap member facing an inward facing side of the main member so as to form a pocket between the outward facing side of the flap member and the inward facing side of the main member,
the flap member being permanently attached to the main member along a first flap attachment segment of the first chassis side edge and along a second flap attachment segment of the second chassis side edge, the first and second flap attachment segments defining a waist region length from the longitudinal ends of each segment closest to the central transversal axis and to the adjacent back edge or front edge of the outer cover,
wherein the side portion length extends by a longitudinal distance beyond the waist region length in the direction towards the transversal axis.

2. The outer cover according to claim 1, wherein the first and second side attachment segments, and/or the first and second flap attachment segments extend essentially parallel to the longitudinal axis.

3. The outer cover according to claim 1, further comprising a first joint assembly comprising one or more joints connecting the first side portion to the chassis portion along the first side attachment segment, and connecting the flap member to the main member along the first flap attachment segment and
a second joint assembly comprising one or more joints connecting the second side portion to the chassis portion along the second side attachment segment and connecting the flap member to the main member along the second flap attachment segment.

4. The outer cover according to claim 1, comprising a first joint connecting the first side portion to the chassis portion along the first side attachment segment, and connecting the flap member to the main member along the first flap attachment segment and
a second joint connecting the second side portion to the chassis portion along the second side attachment segment, and connecting the flap member to the main member along the second flap attachment segment.

5. The outer cover according to claim 3, wherein each of the joint or joints is a stitched seam, a welded seam, an adhesive seam, an adhesive tape or combinations thereof.

6. The outer cover according to claim 3, wherein the joint or joints extend essentially parallel to the longitudinal axis.

7. The outer cover according to claim 1, wherein the longitudinal distance is no less than 0.50× the waist region length.

8. The outer cover according to claim 1, wherein the longitudinal distance is at least 5 cm.

9. The outer cover according to claim 1, wherein the side portion length is from 20% to 30% of a central outer cover length of the outer cover.

10. The outer cover according to claim 1, wherein the waist region length is less than 15% of a central outer cover length of the outer cover.

11. The outer cover according to claim 1, wherein a waist region of the chassis portion is defined transversally between the first and second flap attachment segments, the waist region having the waist region length, and the waist region of the chassis portion comprises one or more insert fastener areas for detachably fastening an insert to the outer cover.

12. The outer cover according to claim 1, wherein at least one out of the outward facing side of the flap member and the inward facing side of the main member forming the pocket comprises one or more insert fastener areas for detachably fastening an insert to the outer cover.

13. The outer cover according to claim 1, wherein the chassis portion of the outer cover further comprises a first elastic edge element and a second elastic edge element, wherein the first elastic edge element is attached along at least a part of the first chassis side edge and the first elastic edge element is adapted to
gather a first gathered edge segment of the first chassis side edge when the first elastic edge element is in a relaxed condition and the first gathered edge segment is gathered with a gathered extension and;
stretch out the first gathered edge segment of the first chassis side edge when the first elastic element is in an outstretched condition and the first gathered edge segment is flat with an ungathered extension; and
the second elastic edge element is attached along at least a part of the second chassis side edge and the second elastic edge element is adapted to
gather a second gathered edge segment of the second chassis side edge when the second elastic edge element is in a relaxed condition and the second gathered edge segment is gathered with a gathered extension and;
stretch out the second gathered edge segment of the second chassis side edge when the second elastic element is in an outstretched condition and the second gathered edge segment is flat with an ungathered extension.

14. The outer cover according to claim 13, wherein the first gathered edge segment and the first side attachment segment are spaced by a second longitudinal distance, and the second gathered edge segment and the second side attachment segment are spaced by a second longitudinal distance, wherein the second longitudinal distance is no more than 3 cm.

15. The outer cover according to claim 13, wherein the first gathered edge segment follows directly from the first side attachment segment, and the second gathered edge segment follows directly from the second side attachment segment.

16. The outer cover according to claim 13, when the outer cover further comprises a first joint assembly comprising one or more joints connecting the first side portion to the chassis portion along the first side attachment segment, and connecting the flap member to the main member along the first flap attachment segment and a second joint assembly comprising one or more joints connecting the second side portion to the chassis portion along the second side attachment segment, and connecting the flap member to the main member along the second flap attachment segment,
the first joint assembly or first joint overlaps with the first elastic edge element, and the second joint assembly or second joint overlaps with the second elastic edge element, as seen in the thickness direction.

17. The outer cover claim 1, wherein the main member has a first transversal Cloth Percentual Stretch in a region extending transversally between the first side attachment segment and the second side attachment segment, and longitudinally along the side portion length, and the flap member and the main member together have a second transversal Cloth Percentual Stretch in a waist region extending transversally between the first flap attachment segment and the second flap attachment segment and longitudinally along the waist region length, wherein the second transversal Cloth Percentual Stretch is less than the first transversal Cloth Percentual Stretch.

18-28. (canceled)

29. The outer cover according to claim 17, wherein the second transversal Cloth Percentual Stretch is from 30 to 70% of the first transversal Cloth Percentual Stretch.

30. The outer cover according to claim 17, wherein each of the first and the second side portions forms a side region, wherein each side region has a side portion transversal Cloth Percentual Stretch, the side portion transversal Cloth Percentual Stretch being less than the first transversal Cloth Percentual Stretch.

31. The outer cover according to claim 17, wherein the side portion transversal Cloth Percentual Stretch is from 30 to 70% of the first transversal Cloth Percentual Stretch.

32. An absorbent assembly comprising the outer cover according to claim 17, and an absorbent insert detachably attached to the outer cover.

33. (canceled)
